(19) United States
(12) Patent Application Publication
(10) Pub. No.: US 2007/0016274 A1
(43) Pub. Date: Jan. 18, 2007

(54) Gastrointestinal (GI) ablation for GI tumors or to provide therapy for obesity, motility disorders, G.E.R.D., or to induce weight loss

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/170,030

(22) Filed: Jun. 29, 2005

(51) Int. Cl. A61F 2/00

(52) U.S. Cl. 607/101; 606/34

(57) ABSTRACT

Method and system for ablating selected portions of the gastrointestinal (GI) tract to provide therapy or alleviate symptoms for GI tract or liver tumors, obesity, motility disorders, GERD, or to induce weight loss. The ablation procedure may be performed endogastrically via mouth and esophagus in an anesthetized patient. Alternatively, the ablations may be performed epigastrically using laproscopic tools. The ablation may involve one of the following: a) ablating tissues of the gastrointestinal (GI) tract; b) ablating tissues of the liver; c) disruption of pacemaker region of the stomach; d) disruption of muscle layers of gastric wall; e) vagal nerve(s) disruption; f) ablation of endogastric lining for cells that produce the hunger hormone ghrelin; and g) disruption of enteric nervous system. The technology used may be one from a group comprising: 1) Radiofrequency catheter ablation; 2) Radiofrequency ablation using an irrigated tip catheter; 3) Microwave ablation; 4) Cryoablation; 5) High intensity focused ultrasound (HIFU) ablation; and 6) Laser ablation.

This application is related to a co-pending application entitled “Method and system for gastric ablation and gastric pacing to provide therapy for obesity, motility disorders, or to induce weight loss” filed Jun. 29, 2005.

### FIELD OF INVENTION

This invention relates generally to medical ablation of tissues, more specifically to gastrointestinal (GI) tract ablations including ablations of liver, stomach or surrounding tissue to provide therapy for GI tumors, obesity, motility disorders, GERD, or to induce weight loss.

### BACKGROUND

Obesity is a significant health problem in the United States and many other developed countries. Obesity results from excessive accumulation-of-fat in the body. It is caused by ingestion of greater amounts of food than can be used by the body for energy. The excess food, whether fats, carbohydrates, or proteins, is then stored almost entirely as fat in the adipose tissue, to be used later for energy. Obesity is not simply the result of gluttony and a lack of willpower. Rather, each individual inherits a set of genes that control appetite and metabolism, and a genetic tendency to gain weight that may be exacerbated by environmental conditions such as food availability, level of physical activity and individual psychology and culture. Other causes of obesity include psychogenic, neurogenic, and other metabolic related factors.

Obesity is defined in terms of body mass index (BMI), which provides an index of the relationship between weight and height. The BMI is calculated as weight (in Kilograms) divided by height (in square meters), or as weight (in pounds) times 703 divided by height (in square inches). The primary classification of overweight and obesity relates to the BMI and the risk of mortality. The prevalence of obesity in adults in the United States without coexisting morbidity increased from 12% in 1991 to 17.9% in 1998.

Treatment of obesity depends on decreasing energy input below energy expenditure. Treatment has included among other things various drugs, starvation and even stapling or surgical resection of a portion of the stomach. Surgery for obesity has included gastroplasty and gastric bypass procedure. Gastroplasty which is also known as stomach stapling, involves constructing a 15- to 30 mL pouch along the lesser curvature of the stomach. A modification of this procedure involves the use of an adjustable band that wraps around the proximal stomach to create a small pouch. Both gastroplasty and gastric bypass procedures have a number of complications.

This Application discloses use of various types of ablation technologies for ablating the gastrointestinal tract for GI tumors, liver tumors, or for ablating the stomach or adjacent areas from the epigastric side or the endogastric side to provide therapy or alleviate symptoms. The ablation technology may be one or more from a group comprising:

a) Radiofrequency catheter ablation

b) Radiofrequency ablation using irrigated tip catheter

c) Microwave ablation

d) Cryoablation

e) High intensity focused ultrasound (HIFU) ablation; and

f) Laser ablation

The ablation site or structure may be one from a group comprising:

a) Tissues of the gastrointestinal (GI) tract;

b) Tissues of the liver;

c) Disruption of pacemaker region of the stomach;

d) Disruption of muscle layers of gastric wall;

e) Vagal nerve(s) disruption;

f) Ablation of endogastric lining for cells that produce the hunger hormone ghrelin; and

e) Disruption of enteric nervous system.

Background of Gastrointestinal (GI) Physiology and Regulation

Shown in conjunction with FIG. 1, the gastrointestinal (GI) tract is a continuous muscular digestive tube that winds through the body. The organs of the GI tract are the mouth, pharynx (not shown), esophagus 3, stomach 54, small intestine (duodenum 7, jejunum, and ileum), and large intestine (cecum, ascending colon, transverse colon, and descending colon).

The gastrointestinal (GI) tract has a nervous system all its own, which is the enteric nervous system 9. This is shown in conjunction with FIG. 2. It lies entirely in the wall of the gut, beginning in the esophagus 3 and extending all the way to the anus. The enteric nervous system has about 100 million neurons, almost exactly equal to the number in the entire spinal cord. It especially controls gastrointestinal movements and secretion. The enteric nervous system is composed mainly of the two plexuses, 1) the myenteric plexus 10, which is the outer plexus lying between the longitudinal and circular muscle layers, and 2) the submucosal plexus 11 that lies in the submucosa. The nervous connection within and between these two plexuses is depicted in FIG. 2. The myenteric plexus controls mainly the gastrointestinal movements, and the submucosal plexus controls mainly gastrointestinal secretion and local blood flow. As also depicted in FIG. 2, the sympathetic and parasympathetic fibers connect with the myenteric 10 and the submocosal 11 plexus. Although the enteric nervous system can function on its own, stimulation by the parasympathetic 12 and sympathetic 13 systems can further activate or inhibit gastrointestinal functions. The autonomic nerves influence the functions of the gastrointestinal tract by modulating the activities of neurons of the enteric nervous system 9.

Shown in conjunction with FIG. 2, sympathetic innervation of the gastrointestinal tract is mainly via postganglionic adrenergic fibers whose cell bodies are located in pre-vertebral and parabertabral ganglia. The celiac, superior and inferior mesenteric, and hypogastric plexus provide sympathetic innervation to various segments of the GI tract. Activation of the sympathetic nerves usually inhibits the motor and secretory activities of the GI system.

Parasympathetic innervation of the GI tract down to the level of the transverse colon is provided by branches of the vagus nerves (10^{th} cranial nerve). Excitation of parasympathetic nerves usually stimulates the motor and secretory activities of the GI tract.

The stomach 54 is richly innervated by extrinsic nerves and by the neurons of the enteric nervous system 9. Axons from the cells of the intramural plexus innervate smooth muscle and secretory cells.

The emptying of gastric contents is regulated by both neural and hormonal mechanisms. The duodenal and jejunal mucosa contain receptors that sense acidity, osmotic pressure, certain fats and fat digestion products, and peptides and amino acids The chyme that leaves the stomach is usually hypertonic and it becomes even more hypertonic because of the action of the digestive enzymes in the duodenum. Gastric emptying is slowed by hypertonic solutions in the duodenum, by duodenal pH below 3.5, and by the presence of amino acids and peptides in the duodenum, The presence of fatty acids or monoglycerides (products of fat digestion) in the duodenum also dramatically decreases the rate of gastric emptying.

Parasympathetic innervation to the stomach is supplied by the vagus nerves, while sympathetic innervation to the stomach is provided by the celiac plexus. In general, parasympathetic nerves stimulate gastric smooth muscle motility and gastric secretions, whereas sympathetic activity inhibits these function. Numerous sensory afferent fibers leave the stomach in the vagus nerves; some of these fibers travel with sympathetic nerves. Other sensory neurons are the afferent links between sensory receptors and the intramural plexuses of the stomach. Some of these afferent fibers relay information intragastric pressure, gastric distention, intragastric pH, or pain.

Shown in conjunction with FIG. 3 is the fundus 15, the body 17, and antrum 19 of the stomach 54. After eating, when a wave of esophageal peristalsis begins, a reflex causes the LES to relax. This relaxation of the LES is followed by receptive relaxation of the fundus 15 and body 17 of the stomach. The stomach 54 will also relax if it is filled directly with gas or liquid. The nerve fibers in the vagi are a major efferent pathways for reflex relaxation of the stomach 54.

FIG. 4 depicts the three main muscle layers of the stomach 54, which are the longitudinal layer 14, the circular layer 16, and the oblique layer 18. The complex and coordinated activity of these muscle layers is responsible for the normally efficient gastric motility. Whereas, the gastric pacing disclosed here from around the antral area of the stomach 54, disrupts the normal gastric motility.

Normally, the smooth muscle of the GI tract is excited by almost continual slow, intrinsic. electrical activity along the membranes of the muscle fibers. This activity has two basic types of electrical waves: 1) slow waves and 2) spikes. This is shown in conjunction with FIG. 5. Most gastrointestinal contractions occur rhythmically, and this rhythm is determined mainly by the frequency of the slow waves of the smooth muscle membrane potential. Their intensity usually varies between 5 and 15 millivolts, and their frequency ranges in different parts of the human gastrointestinal tract between 3 and 12 per minute. The rhythm of contraction of the body of the stomach is about 3 per minute (and in the duodenum is about 12 per minute).

The electrical activity of the GI tract is shown in conjunction with FIG. 5. For example, the contraction of small intestinal smooth muscle occurs when the depolarization caused by the slow wave exceeds a threshold for contraction. When depolarization of a slow wave exceeds the electrical threshold, a burst of action potentials 19 occurs. The action potentials elicit a much stronger contraction than occurs in the absence of action potentials. The contractile force increases with increasing number of action potentials.

Action potentials in gastrointestinal smooth muscle are more prolonged (10 to 20 msec) than those of skeletal muscle and have little or no overshoot. The rising phase of the action potentials is caused by ion flow through channels that conduct both Ca⁺⁺ and Na⁺ and are relatively slow to open. Ca⁺⁺ that enters the cell during the action potential helps to initiate contraction.

When the membrane potential of gastrointestinal smooth muscle reaches the electrical threshold, typically near the peak of a slow wave, a train of action potentials (1 to 10/sec) is fired. The extent of depolarization of the cells and the frequency of action potentials are enhanced by some hormones and paracrine agonists and by compounds liberated from excitatory nerve endings. Inhibitory hormones and neuroefector substances hyperpolarize the smooth muscle cells and may diminish or abolish action potential spikes.

Slow waves that are not accompanied by action potentials elicit weak contractions of the smooth muscle cells (FIG. 5). Much stronger contractions are evoked by the action potentials that are intermittently triggered near the peaks of the slow waves. The greater the frequency of action potentials that occur at the peak of a slow wave, the more intense is the contraction of the smooth muscle. Because smooth muscle cells contract rather slowly (about one tenth as fast as skeletal muscle cells), the individual contraction caused by each action potential in a train do not cause distinct twitches; rather, they sum temporally to produce a smoothly increasing level of tension (FIG. 5).

Between trains of action potentials the tension developed by gastrointestinal smooth muscle falls, but not to zero. This nonzero resting, or baseline, tension of smooth muscle is called tone. The tone of gastrointestinal smooth muscle is altered by neuroeffectors, hormones, paracrine substances, and drugs.

Control of the contractile and secretory activities of the gastrointestinal tract involves the central nervous system, the enteric nervous system, and hormones and paracrine substances. The autonomic nervous system typically only modulates the patterns of muscular and secretary activity; these activities are controlled more directly by the enteric nervous system.

In the current invention, ablation of stomach (gastric) wall or surrounding tissue such as nerve tissue renders the stomach to empty less efficiently. This causes a general feeling of “fullness”, and the patients are not as hungry, which ultimately results in weight loss.

### PRIOR ART

U.S. Pat. No. 6,427,089 (Knowlton) is generally directed to using microwave energy to modifying the stomach wall of a patient.

U.S. patent application publication no. 2004/0181178 (Aldrich et al.), application Ser. No. 10/389,236 is generally directed to use of transesophageal delivery of energy to interrupt the function of vagal nerves.

U.S. patent application publication no. 2004/0215180 (Starkbaum et al.), application Ser. No. 10/424,010 is generally directed to ablation of mucosal tissue to inhibit ghrelin production.

U.S. patent application publication no. 2005/0096638 (Starkbaum et al.), application Ser. No. 10/699,207, is generally directed to ablating tissue from an exterior surface of a stomach.

### SUMMARY OF THE INVENTION

In the method and system of this invention ablation is performed to provide therapy or alleviate symptoms for at least one of gastrointestinal tumors, liver tumors, obesity, motility disorders, GERD, or to induce weight loss, using one of various ablation methods.

Accordingly, it is one object of the invention, to ablate tumor tissues of the gastrointestinal (GI) tract using one of radiofrequency catheter ablation, radiofrequency catheter ablation using irrigated tip catheter, microwave ablation, cryoablation, high intensity focused ultrasound ablation, or laser ablation.

It is another object of the invention, to ablate tumor tissues of the liver using one of radiofrequency catheter ablation, radiofrequency catheter ablation using irrigated tip catheter, microwave ablation, cryoablation, high intensity focused ultrasound ablation, or laser ablation.

It is another object of the invention, to disrupt pacemaker region of the stomach using one of radiofrequency catheter ablation, radiofrequency catheter ablation using irrigated tip catheter, microwave ablation, cryoablation, high intensity focused ultrasound ablation, or laser ablation.

In one aspect ablations to the pacemaker region of the stomach may be performed using endogastric approach.

In one aspect ablations to the pacemaker region of the stomach may be performed using epigstric approach.

It is another object of the invention, to ablate the muscle layers of gastric wall, using one of radiofrequency catheter ablation, radiofrequency catheter ablation using irrigated tip catheter, microwave ablation, cryoablation, high intensity focused ultrasound ablation, or laser ablation.

It is another object of the invention, to ablate vagus nerve(s) tissues around the esophagus or lessor curvature of the stomach using one of radiofrequency catheter ablation, radiofrequency catheter ablation using irrigated tip catheter, microwave ablation, cryoablation, high intensity focused ultrasound ablation, or laser ablation.

It is another object of the invention, to ablate endogastric lining of the stomach using one of radiofrequency catheter ablation, radiofrequency catheter ablation using irrigated tip catheter, microwave ablation, cryoablation, high intensity focused ultrasound ablation, or laser ablation.

It is yet another object of the invention, to ablate nerve tissue of the enteric nervous system using one of radiofrequency catheter ablation, radiofrequency catheter ablation using irrigated tip catheter, microwave ablation, cryoablation, high intensity focused ultrasound ablation, or laser ablation.

These and other objects are provided by one or more of the embodiments described below.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a diagram showing general anatomy of the gastrointestinal (GI) tract.

FIG. 2 is a diagram showing control of the enteric nervous system by the autonomic nervous system (parasympathetic and sympathetic).

FIG. 3 is a diagram showing general anatomy of the human stomach.

FIG. 4 is a diagram showing the longitudinal, circular, and oblique muscle layers of the stomach.

FIG. 5 is a diagram depicting the electrical activity of the GI tract.

FIG. 6 depicts epigastric approach to ablation utilizing laproscopic surgery.

FIG. 7 depicts endogastric approach to ablation via the mouth and esophagus.

FIG. 8A is a simplified block diagram of a radiofrequency ablation system.

FIG. 8B shows a ground patch, and ground patch placement for radiofrequency ablation.

FIG. 9A depicts an area of resistive heating for radiofrequency ablations.

FIG. 9B depicts temperature regions for gastric ablations.

FIG. 10A is a simplified schematic for radiofrequency ablation generator, showing voltage, current, and temperature monitoring.

FIG. 10B is a simplified schematic showing power and impedance as derivatives of voltage and current.

FIG. 11 is a simplified schematic showing the display elements of a radiofrequency ablation generator.

FIG. 12 is a simplified block diagram showing the elements of a microwave ablation system.

FIG. 13 is a diagram showing the principle of high intensity focused ultrasound (HIFU).

FIG. 14 is a simplified block diagram of an ultrasound hyperthermia system.

FIG. 15 is a simplified block diagram of housing for an ultrasound applicator.

FIG. 16 depicts catheter end of an ultrasound ablation system.

FIG. 17 depicts catheter end of a cryoablation probe.

FIG. 18 is cross-section of cryoablation probes.

FIG. 19 is a diagram showing the elements of a laser ablation system.

FIG. 20 is a simplified diagram showing the principle of a laser ablation system.

FIG. 21 is a simplified block diagram showing the elements of a laser ablation system.

FIG. 22 is a simplified diagram of a laser ablation system.

FIG. 23 is a diagram depicting the gastrointestinal tract.

FIG. 24 is a diagram of the stomach showing areas for ablation from the endogastric side and epigastric side.

FIG. 25 is a diagram of the stomach showing areas for ablation from the epigastric side.

FIG. 26 is a graph showing the levels of ghrelin during the day, corresponding with breakfast B, lunch L, and dinner D.

FIG. 27A is a diagram showing an ablation catheter in the esophagus, near the lower esophageal sphincter.

FIG. 27B is a diagram of the ablation catheter of FIG. 27A.

FIGS. 28A, 28B, and 28C depict ablation in the lower esophageal region.

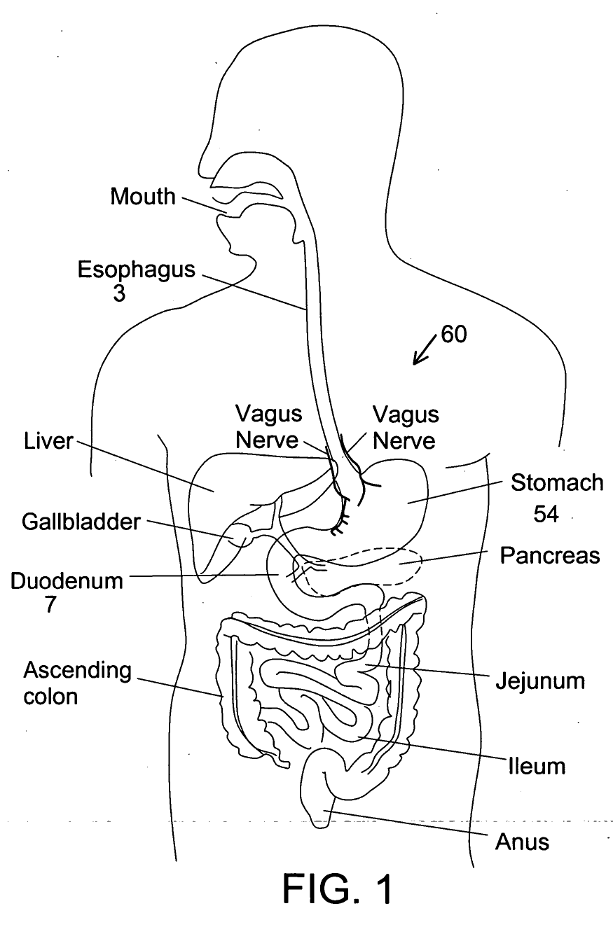

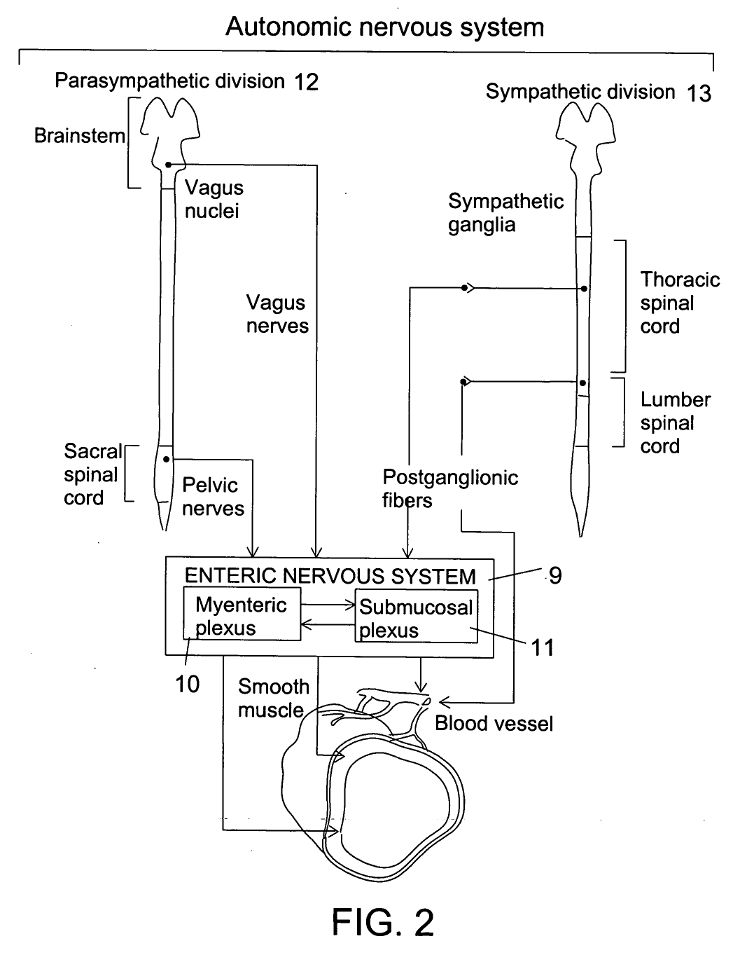

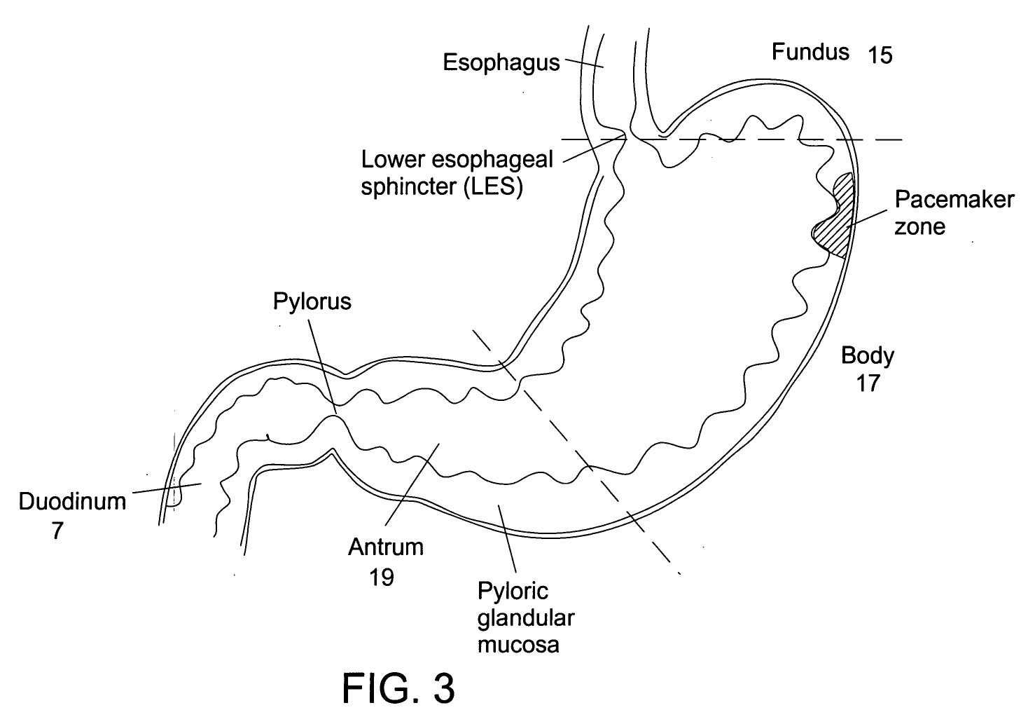

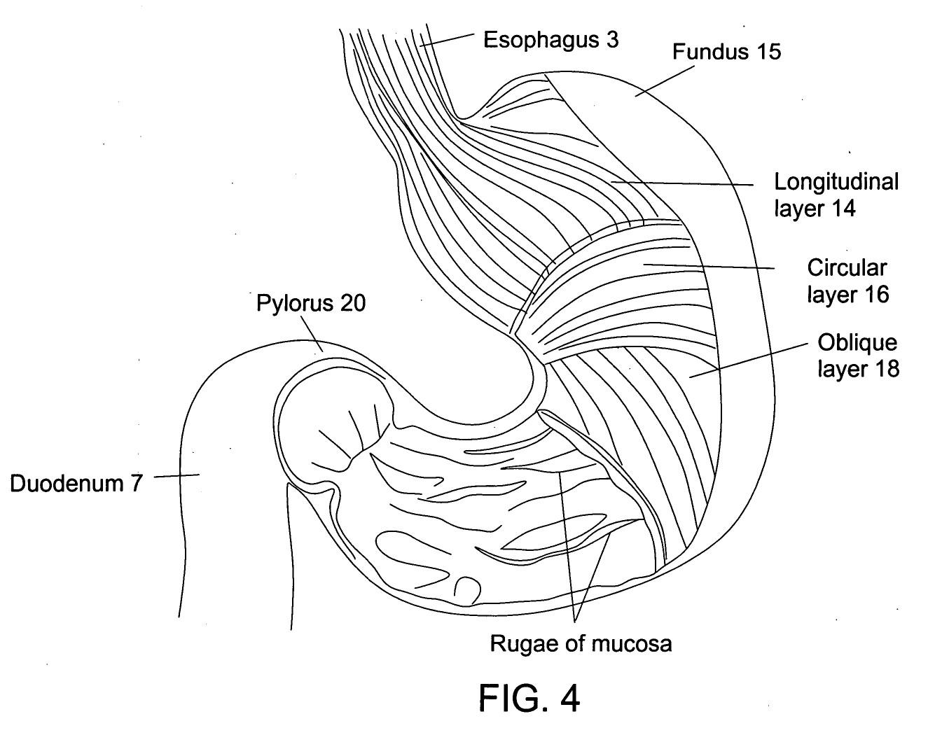

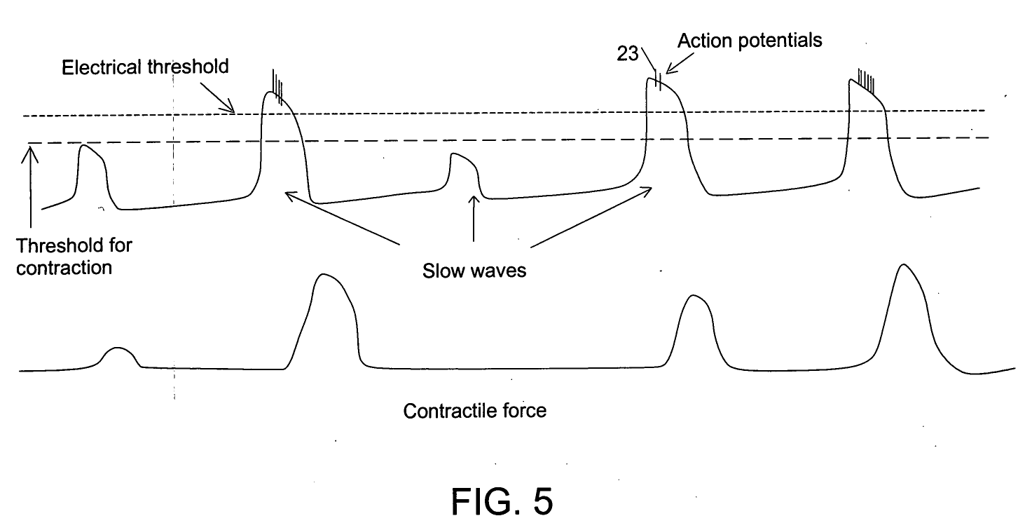

### DESCRIPTION OF THE INVENTION

In the method and system of this invention, ablation of stomach and/or other parts of the gastrointestinal (GI) tract is performed to provide therapy for at least one of gastrointestinal tumors, liver tumors, obesity, to induce weight loss, as well as other gastrointestinal (GI) disorders.

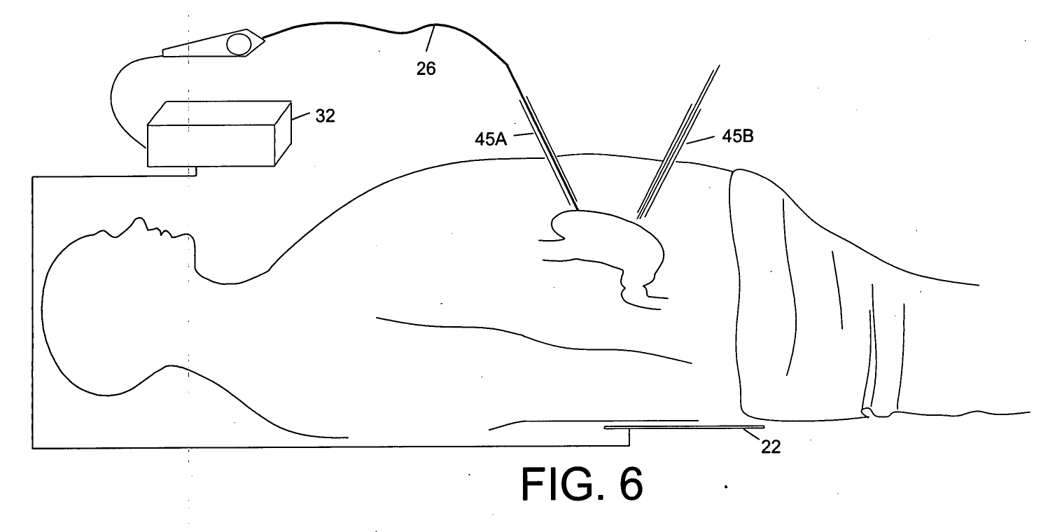

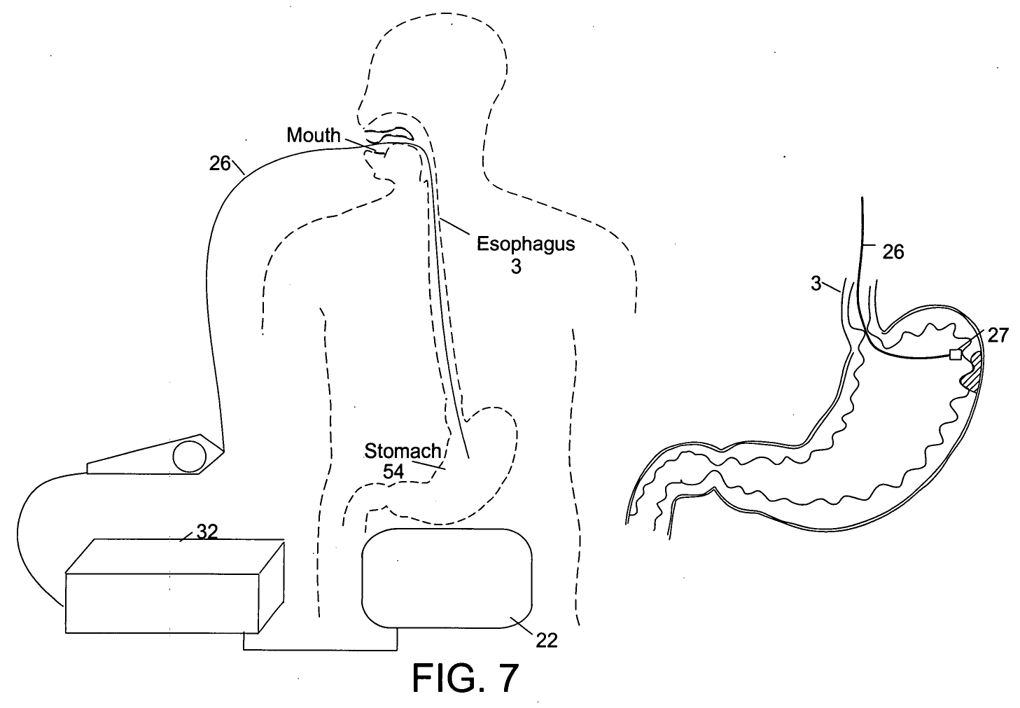

The ablation of stomach may be performed from the epigastric side (shown in FIG. 6) or from endogastric side within the stomach wall (shown in FIG. 7). Referring to FIG. 6, for epigastric ablation, the ablation catheter is inserted into the abdominal cavity laproscopically, and ablation lesions are performed on the epigastric surface of the stomach. For performing the ablation procedure, using the epigastric approch, the patient is positioned in the lithotomy position and anesthetized. The abdomen is cleansed with an antiseptic solution and draped in a sterile fashion. The trocars 45A, and 45B are inserted. One trocar 45A is needed for introducing the ablation catheter 26. A second trocar 45B is needed for introducing the optical system. An optional third trocar can be used to introduce a liver retractor.

After retracting the liver, the optical system is used for identifying the anatomical structure to be ablated. Different forms of ablation energies may used, such as radiofrequency (RF) catheter ablation, RF ablation with an irrigated tip catheter, microwave ablation, high intensity focused ultrasound (HIFU) ablation, cryoablation, and laser ablation. These are further described later in this disclosure.

Alternatively, the ablation may be performed from the endogastric side (FIG. 7). Combination of epigastric and endogastric ablations may also be performed. As one example without limitation, a patient may have epigastric ablation procedure performed, and at a later date may have endogastric ablation procedure, or vice versa.

As shown in conjunction with FIG. 7, for endogastric ablations, the ablation catheter 26 may be introduced in an anesthetized patient, via the mouth and esophagus, and positioned at the appropriate site within the stomach 54. Even though FIG. 7 is shown in reference to radiofrequency (RF) ablation, other forms of ablation energies may also be used such as RF with irrigated tip catheter, microwave energy, cryoablation, high intensity focused ultrasound (HIFU) ablation, cryoablation, and laser ablation.

Table one below, summarizes the ablation technology that may be used, as well as, the ablation site or structure at or around the stomach, such as the nerve plexus that carry hunger or satiety signals to the brain.

TABLE 1

| ABLATION TECHNOLOGY | | ABLATION SITE OR STRUCTURE | |
|---|---|---|---|
| 1. | Radiofrequency catheter | 1. | Tumor issues of the |
| | ablation | | gastrointestinal (GI) tract |
| 2. | Radiofrequency irrigated tip | 2. | Tumor tissues of the liver |
| | catheter ablation | | |
| 3. | Microwave ablation | 3. | Disruption of pacemaker region |
| | | | of the stomach |
| 4. | Cryoablation | 4. | Disruption of muscle layers of |
| | | | gastric wall |
| 5. | High intensity focused | 5. | Vagal nerve(s) disruption |
| | ultrasound (HIFU) ablation | | |
| 6. | Laser ablation | 6. | Ablation of endogastric lining- |
| | | | for cells that produce the |
| | | | hunger hormone ghrelin |
| | | 7. | Disruption of enteric nervous |
| | | | system |

In the method of this disclosure, the following combinations can be practiced:

1) Ablation of gastrointestinal (GI) tissues using, a) Radiofrequency catheter ablation, b) Radiofrequency catheter ablation using irrigated tip catheter, c) Microwave ablation, d) Cryoablation, e) High intensity focused ultrasound ablation, and f) Laser ablation.

2) Ablation of Liver tissues or liver tumors using, a) Radiofrequency catheter ablation, b) Radiofrequency catheter ablation using irrigated tip catheter, c) Microwave ablation, d) Cryoablation, e) High intensity focused ultrasound ablation, and f) Laser ablation.

3) Disruption of pacemaker region of the stomach using, a) Radiofrequency catheter ablation, b) Radiofrequency catheter ablation using irrigated tip catheter, c) Microwave ablation, d) Cryoablation, e) High intensity focused ultrasound ablation, and f) Laser ablation.

4) Disruption of muscle layers of gastric wall, using a) Radiofrequency catheter ablation, b) Radiofrequency catheter ablation using irrigated tip catheter, c) Microwave ablation, d) Cryoablation, e) High intensity focused ultrasound ablation, and f) Laser ablation.

5) Vagus nerve disruption using, a) Radiofrequency catheter ablation, b) Radiofrequency catheter ablation using irrigated tip catheter, c) Microwave ablation, d) Cryoablation, e) High intensity focused ultrasound ablation, and f) Laser ablation.

6) Ablation of endogastric lining—for cells that produce the hunger hormone ghrelin using, a) Radiofrequency catheter ablation, b) Radiofrequency catheter ablation using irrigated tip catheter, c) Microwave ablation, d) Cryoablation, e) High intensity focused ultrasound ablation, and f) Laser ablation.

7) Disruption of enteric nervous system using, a) Radiofrequency catheter ablation, b) Radiofrequency catheter ablation using irrigated tip catheter, c) Microwave ablation, d) Cryoablation, e) High intensity focused ultrasound ablation, and f) Laser ablation.

### Radiofrequency Ablation

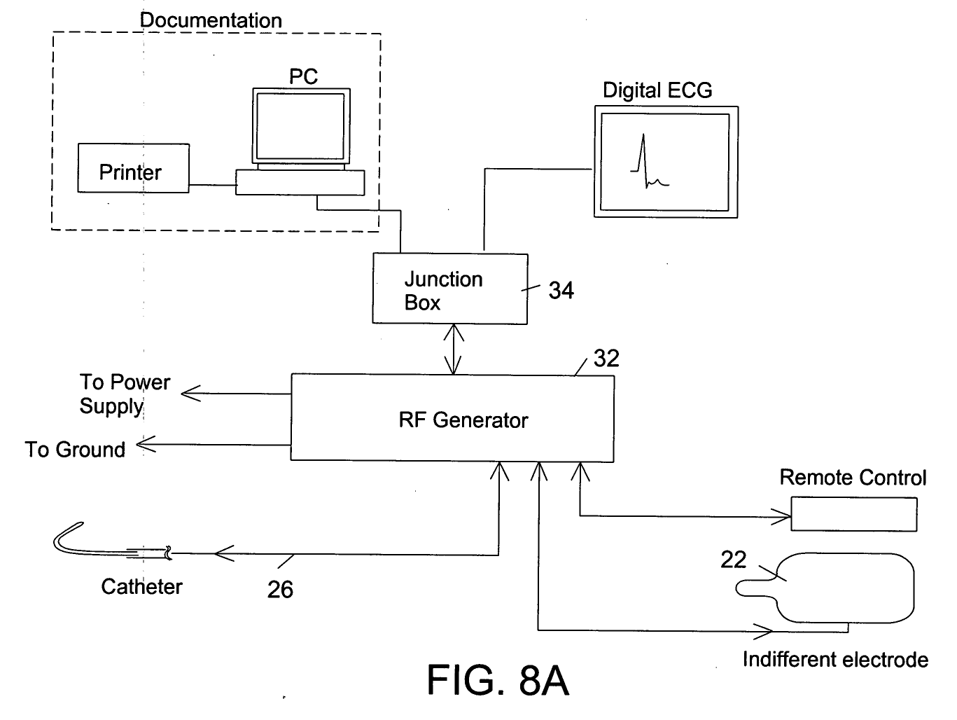

RF ablation is shown with reference to FIGS. 8A and 8B. The RF generator 32 is a source of RF voltage between two electrodes. When the generator is connected to the tissue to be ablated, current will flow through the tissue between the active and dispersive electrodes. The active electrode is connected to, for example the stomach tissue (or tissue surrounding the stomach) where the ablation is to be made, and the dispersive electrode 22 is a large-area electrode forcing a reduction in current density in order to prevent tissue heating. The return electrode (dispersive) (ground pad) 22, is needed to induce resistive heating. Without it the system would look like an open circuit, and there would not be any current flowing through the target tissue and thus there would be no heating. As shown in conjunction with FIG. 8B, the relatively large dispersive electrode 22 would be typically placed on the lower back of a patient, but can be placed at other sites.

When using radiofrequency (RF) ablation, the total RF current, IRF is a function of the applied voltage between the electrodes connected to the tissue, and the tissue conductance. The heating distribution is a function of the current density. The greatest heating takes place in regions of the highest current density, J. The mechanism of tissue heating in the RF range of hundreds of KHz is primarily ionic. The electrical field produces a driving force on the ions in the tissue electrolytes, causing the ions to vibrate at the frequency of operation. The current I density J=σE, where σ is the tissue conductivity. The ionic motion and friction heats the tissue, with a heating power per unit volume equal to J2/σ. The equilibrium temperature distribution as a function of distance from the electrode tip, is related to the power deposition, the thermal conductivity of the target tissue, and the heat sink which is a function of blood circulation. The lesion size, is in turn, a function of the volume temperature. Many theoretical models to determine tissue ablation volume as a function of tissue type are available. In RF ablation, lesion formation results from resistive tissue heating at the point of contact with the RF Electrode. This heating leads to coagulation necrosis and permanent tissue damage. If there is poor tissue contact, RF current can not be coupled to the underlying tissue, and the desired effect of tissue heating is lost.

Radiofrequency ablation applies an alternating current to tissue, in the range of 300 to 1 MHz (typically in the 500-KHz frequency range). Unlike direct current, which creates cellular injury via electrolytic dissociation of tissue fluids, alternating current causes tissue damage from heat via protein denaturation, blood coagulation, and fluid evaporation. It is similar to electrocautery but generally less destructive because of the larger surface area of the surgical probe, and the regulation of power delivery via probe thermistor measurement of tissue temperature.

### Mechanism of Tissue Heating

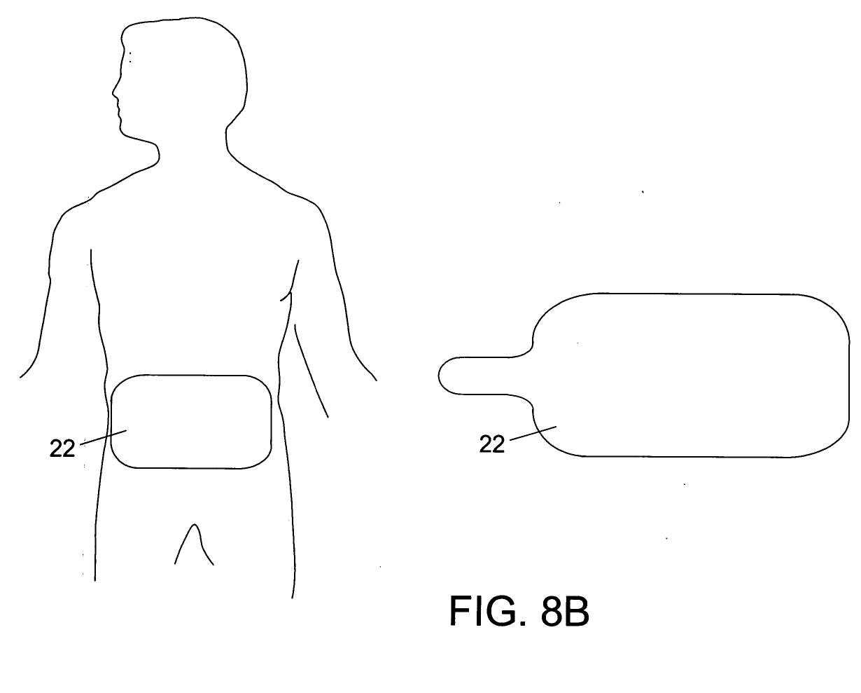

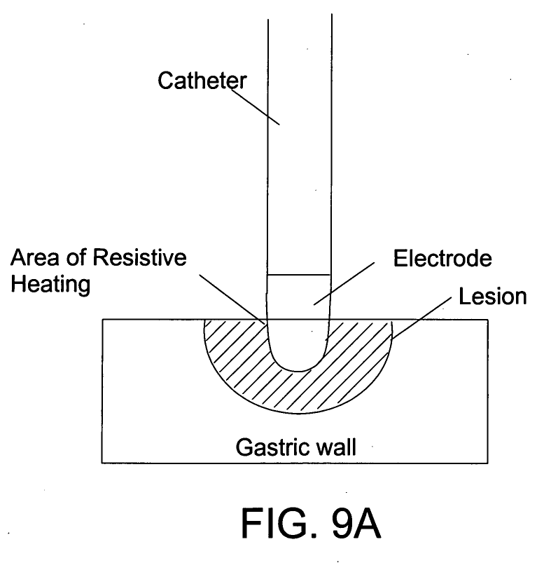

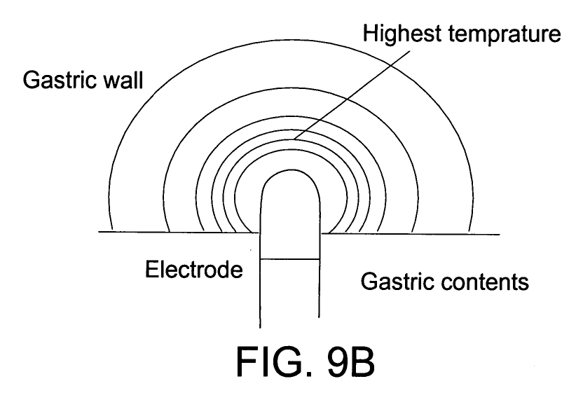

Shown in conjunction with FIGS. 9A and 9B, radiofrequency energy heats tissue in two main ways. First, ohmic heating occurs (FIG. 9A) on the surface by a mechanism in which the gastric tissue in direct contact with the coil or probe acts as a resistor. This heating falls off by the fourth power of distance from the electrode in unipolar systems and typically penetrates only 1 mm. Second, conductive heating occurs (depicted in FIG. 9B), in which this surface heat is transferred to increasingly deeper tissue; conductive heating accounts for the majority of the lesion depth.

RF can be applied either in unipolar (as was depicted in FIGS. 9A and 9B) fashion from a tissue electrode source to grounding pad serving as the indifferent electrode 22, or between two bipolar tissue electrodes. Bipolar RF systems intended for surgical use apply two linear electrodes that gently squeeze together on either side of the gastric tissue. This creates two opposing surfaces of ohmic heating and improves the efficiency with which the conductive heating occurs.

### Determinants of RF Lesion Size

When using RF ablation, the RF electrode temperature is a better predictor of RF lesion size than delivered energy or current. Monitoring of electrode temperature is typically carried out with one or more thermistors. The maximal lesion size from conductive heating is determined primarily by the electrode surface area and electrode-tissue contact temperature, and is achieved at a rate that is a reverse exponential decay with half-time of 7 to 9 seconds.

Lesion size is also influenced by time, irrigation of the electrode, impedance rise, and convective cooling. The duration of energy delivery has a diminishing effect on reaching maximal lesion size after 20 seconds. Electrode irrigation results in deeper lesions. Impedance rises with increased power, increased electrode-tissue pressure, and repeat applications. Saline is protective against impedance rises when compared to blood.

The Bostom Scientific/EP Technologies Cobra system (San Jose, Calif.) is one radiofrequency system approved for commercial use in the United States for general surgical tissue ablation, and may be used for the methods of this invention. The electrosurgical unit (ESU) generates a 500 kHz sine wave. This surgical probe is a flexible single-use probe consisting of seven coagulating electrodes; six of the seven are 12.5 mm coiled electrodes spaced 2 mm apart, and the seventh is an 8 mm distal-tip electrode. Active coils are selected on the ESU prior to the delivery of each lesion. Two skin grounding pads are required to serve as indefferent electrodes.

Finite element simulation of RF ablation using these coil electrodes shows maximal current density at the coil ends, with 2 mm extension of the 50° C. tissue heat isotherm from the coil ends. Each electrode coil contains two temperature-sensing thermistors. One is located 1800 apart at each coil end, where resistive heating is greatest. In vitro testing at 80° C. has shown all lesions from adjacent coils to be contiguous, although this is only true in 75% of lesions made at 70° C.

### Electrode and Catheter

To deliver power more efficiently, material which has better thermal conductivity can be chosen. It has been shown that gold, which has four times the thermal conductivity of platinum yields a larger lesion.

The electrode can be designed to cool the tip, thus avoiding tissue charring. The cool-tip catheter using chilled water is one example. Because charring can be avoided. Power can be delivered for a longer time thus allowing the conduction to be carried deeper, thereby increasing the lesion depth. One possible problem with the cool-tip method is the inability to precisely determine the maximum temperature since the maximum temperature is located beyond the cooled electrode surface.

The electrode tip diameter has generally been increased to obtain wider lesions and to allow cooling by nearby fluid flow, thus creating deeper lesions as well. The larger tip diameter, however, creates the need to control nonuniform heating and the presence to hot spots.

Phased RF ablation allows usage of multiple electodes on the same or different catheters. Because adjacent electrodes are in different phases with respect to each other, an RF signal is applied uniformly such that there will be a voltage gradient between electrodes thus creating bipolar heating simultaneously. The advantages of these RF methods include an increase in uniform heating and the possibility to create long, linear lesions, which is useful for gastric muscle lesions.

Balloon electrode RF ablation is another method for a larger tip diameter while still having the ability to be percutaneously inserted. It uses a semipermeable and conductive membrane, such as gold foil, that is inflated with saline when the catheter is inside. Dominant heating occurs at the interface of the balloon and the tissue.

RF electrode design can also use a gel or electrolytic solution, such as saline, instead of direct contact between the metal electrode and the tissue. This produces a more even heat distribution in the tissue. In the design of the electrode for soft tissue shrinkage, the electrolytic solution is cooled to about 30 to 55° C. Not only does this electrolytic solution provide electrical conduction, it also has a cooling effect to avoid too high a temperature at the interface of the electrode and the tissue. Gold coating has been used to prevent corrosion in the saline envirnment. Saline can also be a choice for an irrigation solution because it has the same concentration as the body's fluids, this it is not absorbed by the body.

Having a shaft that can bend 90° can be useful for accessing the back of a joint or the mouth while a bend of 10 to 30° is good for the front part of a joint compartment or the mouth or nose.

### Active Electrode Cooling RF Ablation for Obtaining Deeper Lesion

The cooling goal can be obtained with either cool water as in the Cool-tip method or with saline. This method produces a more uniform temperature distribution and allows a longer power delivery thus obtaining a larger lesion without tissue desication.

### Ablation Generator

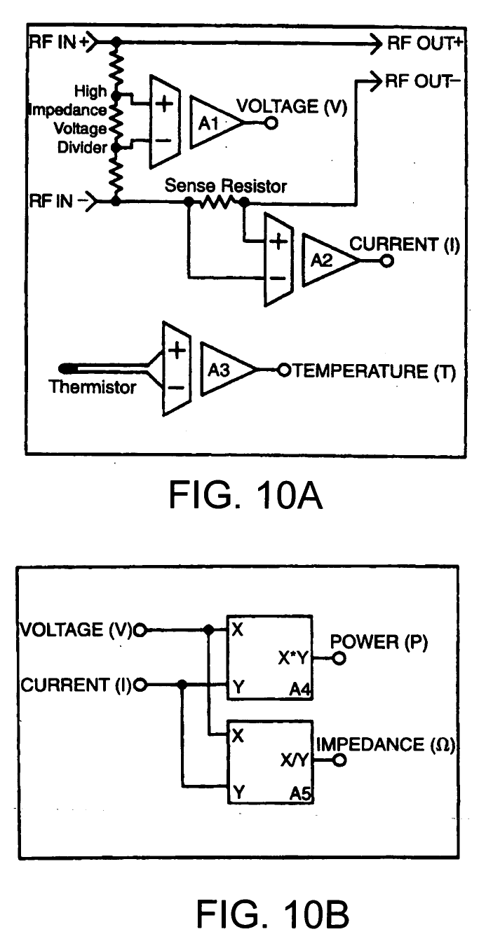

FIGS. 10A and 10B describe in simplified block diagrams the circuitry used to monitor the appropriate radiofrequency parameters. Voltage, current, and temperature are generally measured, and all other parameters are generated. As shown in FIG. 10A, Amplifier A1 uses a high-impedance voltage divider to measure a fraction of the radiofrequency voltage across the outputs. This is isolated, converted to a root-mean-square (RMS) value, and scaled to an appropriate level. The RMS signal has a very-low-frequency waveform and can be easily displayed or digitized at low sampling intervals. Amplifier A2 samples the radiofrequency current by using the current sensing resistor, or a coil can be placed around the return. This signal is also isolated, converted to an RMS value, and scaled. A thermistor is used to measure temperature at the catheter tip. Amplifier A3 isolates the signal, converts the change in resistance to a linearized voltage, and scales the output. The thermistor placement is critical to correct temperature monitoring. This sensor is usually placed as close to the tip as possible and thermally isolated from the rest of the electrode. Even with these precautions, the temperature that is monitored by the system is only an approximation of the tissue temperature at the lesion site. The electrode temperature that is recorded represents a complex interaction of heat generated in the tissue interface, the radiofrequency field, and convective heat loss to surrounding blood and tissue. Although not ideal, it is the best system available.

The signals for power and impedance are derived from the measured values of voltage and current. Given a sinusoidal signal and assuming resistive loads as the major component affecting the output, the following relationships can be used:
Impdence=Voltage/current
Power=Voltage×Current

These associations can be generated by using analog computational blocks as shown in FIG. 10B or by mathematically processing digitized signals.

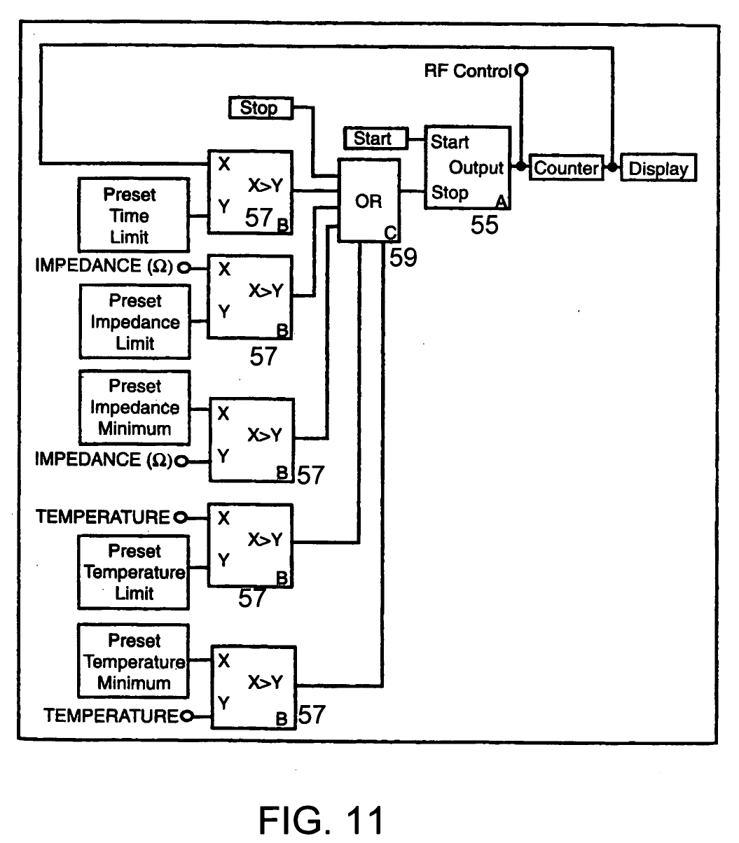

When a generator's output is started or terminated depends on an interaction of the operator and automatic relationships set by the operator or manufacturer. FIG. 11 is a simplified block diagram of a digital control of the generator 32 output. Block A 55 represents a set-reset flip-flop. The output goes true when the start input is set and false when the stop input is set. This output turns on the generator and starts the time counter. Block C 59 is the Boolean OR function and is set true if any of its inputs are true. It serves to sum all of the limit conditions that can stop the generator's output. The B blocks 57 represent comparators, for which the output goes true whenever the X input is greater than the Y input. Otherwise, the output stays false. In this manner, the generator output is terminated whenever the time exceeds the set time, the impedance is outside the set minimum and maximum, the temperature is outside the preset minimum and maximum, or the operator pushes stop

Radiofrequency generator can also operate in a power mode. In this mode of operation, time duration is selected, limits on impedance or temperature are set or predetermined by the manufacture, and the desired power level is chosen. The generator outputs the set level of power while allowing the operator to see how the impedance and temperature levels are changing. If an adequate tip temperature is not reached quickly, the operator can terminate the delivered energy or adjust it. If the safety limits of the temperature or impedance settings are exceeded, automatic shutdown occurs.

Because temperature can be crucial to the success of catheter ablation, a temperature mode of operation has been developed. This is also referred to the closed-loop mode of operation. The rationale is to ensure target-tissue temperatures. Instead of the operator choosing a set power level, a temperature set point is selected. The generator then adjusts the power level and monitors the temperature output. Initially, the power is limited as heating begins. The generator then delivers a much larger output level. Usually the maximum, as long as the difference between the set point and the monitored value is larger (10° to 12° C.) than a manufacturer's determined level. After that difference is at or below the manufacturer's setting, power drops off. When the temperature difference becomes sufficiently small (2° to 3° C.), a minimal amount of power is delivered to maintain temperature and to allow monitoring of other parameters. The generators typically cease to deliver power if any of the safety limits are exceeded.

### Microwave Ablation

In the method and system of this invention, microwave energy may be delivered through a probe or catheter antenna to the affected gastric or surrounding tissue which allows the procedure to be performed percutaneously or endoscopically. In microwave ablation, the frequencies 915 MHz and 2.45 GHz are usually used due to Federal Communications Commission (FCC) restrictions.

Unlike RF which generate lesions of relatively limited size and penetration, microwave energy usually allows for greater tissue penetration, and thus a greater volume of heating. Table two below compares some features of RF vs. microwave ablation.

TABLE TWO
Comparison of RF vs. Microwave

| | Radiofrequency | Microwave |
|---|---|---|
| Waveform | Continuous | N/A |
| | Unmodulated sinusoidal | |
| Frequency | 300-1,000 kHz | 915, 2,450 MHz |
| Voltage V | <100 V | N/A |
| Mechanism of injury | Resistive heating | Radiant heating |
| Lesion size | Small | Unknown |
| Control of injury | High | High |

In microwave ablation, a lesion is created as heat conducts passively away from this zone and the surrounding myocardium is heated to a temperature where cell death occurs (approx. 50° C.). Lesion size is therefore a function of the size of the electrode and the resulting temperature at the electrode tissue interface.

The mechanism of thermal injury in microwave ablation is dielectric heating. Body tissue contains various polar molecules, of which water is the most abundant and has an exceptionally high polarity. At microwave frequencies, electromagnetic radiation causes rotation of molecular dipoles; heat is created as these movements are opposed by intermolecular bonds and thus represents dissipation of the part of the energy of the electromagnetic field in the form of molecular friction. Energy absorption is affected by the presence of electrolytes and other polar molecules such as amino acids in tissue water. Conductive heating is a comparatively minor contributor to tissue heating. Heat is produced by the mechanical friction between the water molecules and surrounding structures.

Microwave hyperthermia has shown to be useful in radiation oncology for the treatment of various solid tumors. Also, because of its experience in enlarging myocardial lesions in catheter ablation, microwave energy would be useful in gastric ablation. Microwave energy is delivered down the length of a coaxial cable that terminates in an antenna capable of radiating the energy into tissue. Radiant energy causes the water molecules in myocardial tissue to oscillate, producing tissue heating and cell death. The higher frequency of microwave energy allows for greater tissue penetration and theoretically a greater volume of heating than that possible with RF, which produces direct ohmic or resistive heating.

Microwave energy for tissue ablation effects has been studied using a helical antenna mounted on a coaxial cable (2.44 mm o.d.). High-frequency current at 2,450 MHz was delivered via the helical antenna into a tissue-equivalent phantom model. The temperature distribution profile was measured around the antenna as well as into surrounding volume (the depth of penetration). The volume of heating for the microwave catheter system was 11 times greater than that of an RF electrode catheter at the same surface temperature. In addition, the microwave catheter penetrated an area that was twice as large as that penetrated by the RF catheter. These data suggest microwave energy will produce larger lesion than RF because a greater volume of tissue is being heated, this is advantageous for gastric ablations. An additional theoretical advantage of the microwave system is that direct tissue contact is not crucial for tissue heating since heating occurs via radiation, and not via direct ohmic heating as seen with RF.

Helical and whip antenna designs have also been evaluated in a tissue-equivalent phantom at 915 MHz and 2,450 MHz utilizing a coaxial cable (0.06 in o.d.). All catheters were measured utilizing a network analyzer prior to placing them in the phantom model. Such analysis demonstrated the great variability in tuning of these microwave catheters.

### Microwave Ablation

In general, higher water content (HWC) means higher dielectic loss and HWC tissues will absorb more energy. Low water content (LWC) tissues, such as fat or bone, have dielectric constants and conductivities about one order of magnitude smaller that high water content (HWC) tissues, such as muscle or organs.

Many of the benefits of microwave ablation relate specifically to its mode of heating. Heating occurs in volume and relies very little on thermal flow, allowing microwaves to ablate areas near high blood flow. This is a distinct advantage over RF ablation. Because of the volume heating effect, charring may be eliminated and simply increasing the applied power will also increase lesion size. Power deposition falls as a function of 1/r³ in microwave ablation (as opposed to 1/r⁴ in RF) so power will theoretically travel farther and more uniformly into the tissue. Serious complications apparent in other ablation modalities have not been seen in microwave ablation. Antennas need only be a few centimeters long, reducing the invasiveness of the procedure. Arrays of probes may be employed to increase lesion size or uniformity. In addition, the probe or catheter antennas may be easily sterilized and reused, reducing procedure costs.

### Microwave Generator

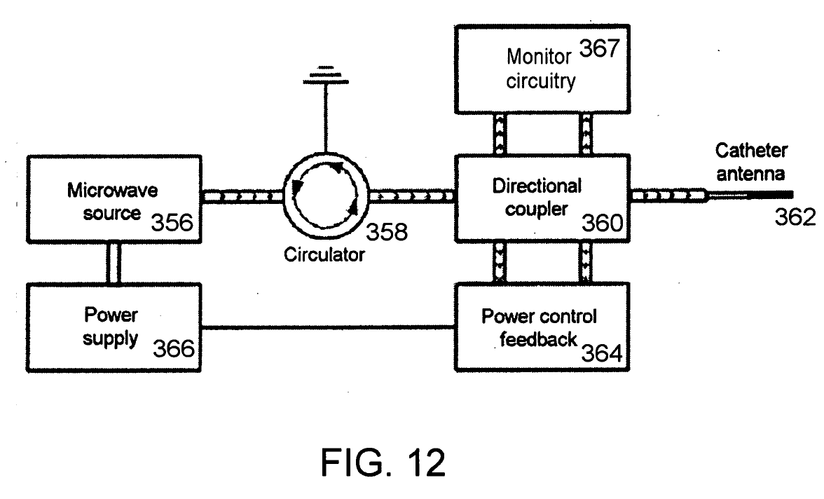

Tissue-ablation microwave generators typically generate the electromagnetic field using a magnetron, such as is used in microwave ovens. The microwave generator provides the necessary microwave power to be delivered to the antenna. Several methods to create this power are available. In general, there are two subcomponents to the generator: a power supply and a microwave source. The power supply converts the line poser (typically 120 VAC, 60 Hz) to a suitable supply for the microwave source. The microwave source then converts the electrical power to microwave power. Shown in conjunction with FIG. 12 is a simplified block diagram of a microwave system, comprising a microwave source 356, coupling network 360, power supply 366, and the catheter antenna 362.

The most common microwave source used in ablation systems is a magnetron due to its low cost, high power output (often several MW), and high conversion efficiency (>80%). The magnetron is a crossed-field resonant cavity tube that converts electron motion to microwave poser. The magnetron filament is heated with a high current (3.3 V, 10 A typical) until thermionic emission causes electrons to “boil” off similar to water molecules boiling off as steam. The high negative potential between the cathode and anode (4 kV typical) creates a large electric field that accelerates the electrons toward the anode. As they accelerate, the axial magnetic field exerts a force on the electrons in a direction perpendicular to their original motion; that is, it pushes the electrons azimuthally around the cathode.

The electric and magnetic field strengths are usually set so that the curving path of an electron just skims the face of the anode block. In this way, the electrons interact with the resonant cavities to set up EM fields. Hence, energy is transferred from the electron motion to the EM fields inside the cavities. Each cavity resonates at the design frequency (2.45 GHz, for example) and a loop is placed inside one of the cavities to extract the microwave power.

The AFx system (AFx, Inc., Freemont, Calif.) is one currently available microwave system available for cardiac tissue ablation. This system may also be adapted to be used for gastric ablations. The system consists of a magnetron-powered 2.45 GHz generator with power and timer settings, and a hand-held surgical probe that has an antenna at the end through which the electromagnetic radiation is emitted. The Flex-2 is a surgical probe with a 2 cm rigid antenna. The Flex-4 probe has both a bendable shaft and a 4 cm flexible antenna. The antennas have the desirable feature of being shielded on one side. This ensures that only one side of the antenna delivers the ablation energy, an advantage for epigastric ablations.

### High Intensity Focused Ultrasound (HIFU) Ablation

In one aspect of the invention, ablations may be performed using high intensity focused ultrasound (HIFU). When high-intensity ultrasound waves are focused at targets deep within the human body, the temperature in the region of focus can be increased to a level high enough to kill the cells in that region.

Ultrasound has several characteristics which make it well suited for the induction of thermal therapy. These include the feasibility of constructing applicators of virtually any shape and size, and good penetration of ultrasound at frequencies where the wavelengths are on the order of millimeters. The small wavelengths allow the beams to be focused and controlled. Clinical research has shown that ultrasound beams can penetrate deep and that the power deposition pattern can be controlled.

Ultrasound is a form of mechanical energy that is unique among available medical radiation methods in that it can be sharply focused within the tissue. The usual frequency range of medical ultrasound used for imaging and surgical application is 0.5 MHz to 20 MHz. For this range, it has a low absorption rate in soft body tissue and a relatively short wavelength. While the absorption rate limits how deeply the wave can travel inside of the body, the wavelength governs how precisely the wave can be focused onto the tissue. Hence, ultrasonic energy can be deposited deep inside the body with precise focus. As the ultrasound pressure wave travels through the body it loses energy due to scattering and absorption. Scattered energy is used for imaging while energy absorption causes tissue heating.

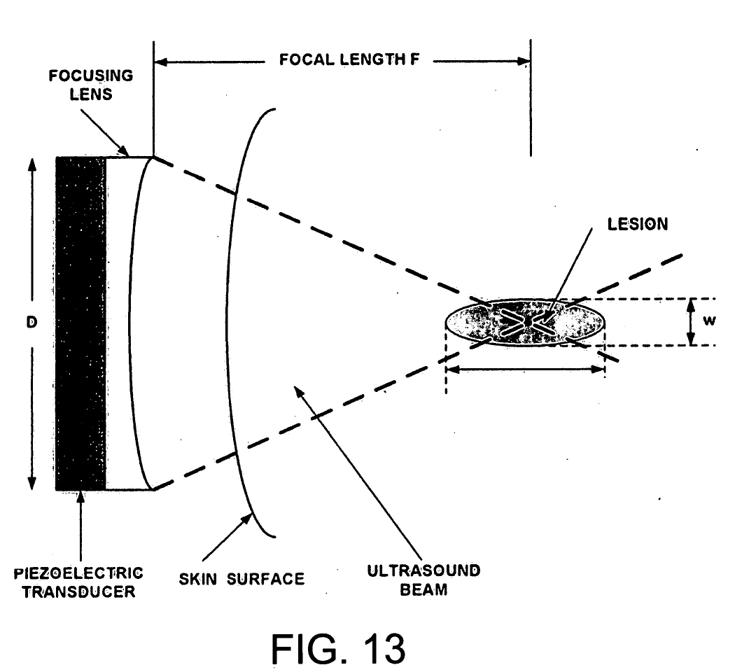

Shown in conjunction with FIG. 13, is the basic principle of the ultrasonic ablation technique which is referred to synonymously as focused ultrasound surgery (FUS) or high-intensity focused ultrasound (HIFU). In this technique, a high-intensity ultrasound beam is brought to a tight focus within the target tissue volume, which may lie deep within the body. The beam passes through the overlying skin and other tissues without harming them. The absorption reaches a maximum in the focal volume where the intensity is at its highest. The temperature at the focal volume is raised to 56° C. and held there for 1 to 3 seconds, which kills the cells in focus. There is a very sharp boundary between dead and live cells at the border of the focal volume. Also shown in FIG. 13, the source is a planar ultrasound transducer with diameter D and is situated outside of the body. The ultrasound beam is focused at the desired depth inside of the body by a focusing lens. The lesion produced has length l and width w and is ellipsoid or cigar shaped.

### Heating Mechanisms and Biological Effects

HIFU produces an effect on tissues by several mechanisms: thermal effects, cavitation, other mechanical forces, and chemical reactions and acceleration. Thermal and cavitation mechanisms are the most important and best understood. Thermal heating is caused by absorption of ultrasonic energy by the tissues. This leads to a rise in temperature of the tissues. Consequently, the rise in temperature is dependent on the intensity of the ultrasound beam and the heat absorption coefficient of the tissue. In HIFU, the ultrasonic intensity at the beam focus is much higher than that outside of the focus. The ultrasonic focus can easily generate temperature elevation of 30° C. to 40° C., coagulating tissue in just a few seconds.

### Ultrasonic Ablation System

A complete HIFU system would normally consist of an ultrasonic applicator, electromechanical components for steering and positioning the acoustic beam, a display for therapy planning and imaging, and a computer for HIFU dosage calculations and control, as well as, for monitoring feedback during ablation.

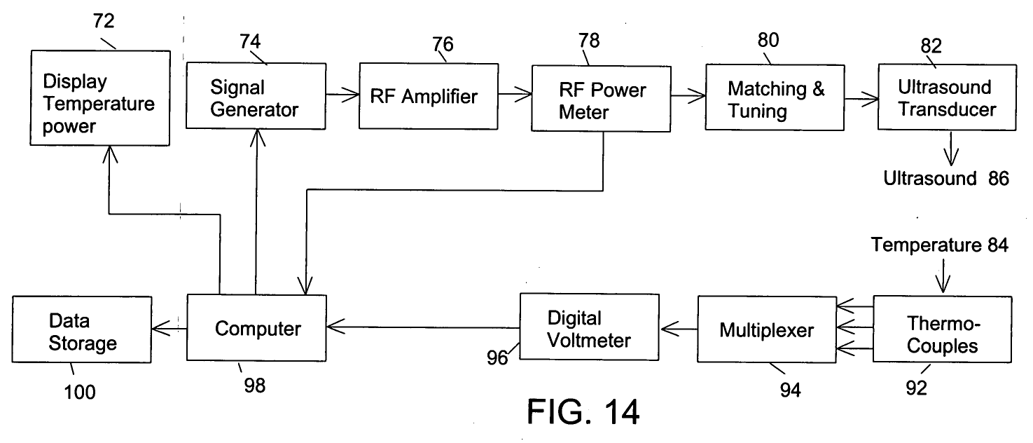

Shown in conjunction with FIG. 14 is a simplified block diagram of an ultrasound system for hyperthermia induction. The RF signal is generated by a signal generator 74 or an oscillator and is amplified by an RF amplifier 76. The generation of the RF signals to be converated into mechanical motion is in principle similar in all systems. The forward and reflected electrical power are measured after amplification in order to obtain the total acoustic power output. The signal enters the transducer through a matching and tuning 80 network that couples the electrical impedance of the transducer 82 to the output impedance of the power amplifier 76. The power output is controlled by the amplitude and duty cycle of the RF voltage.

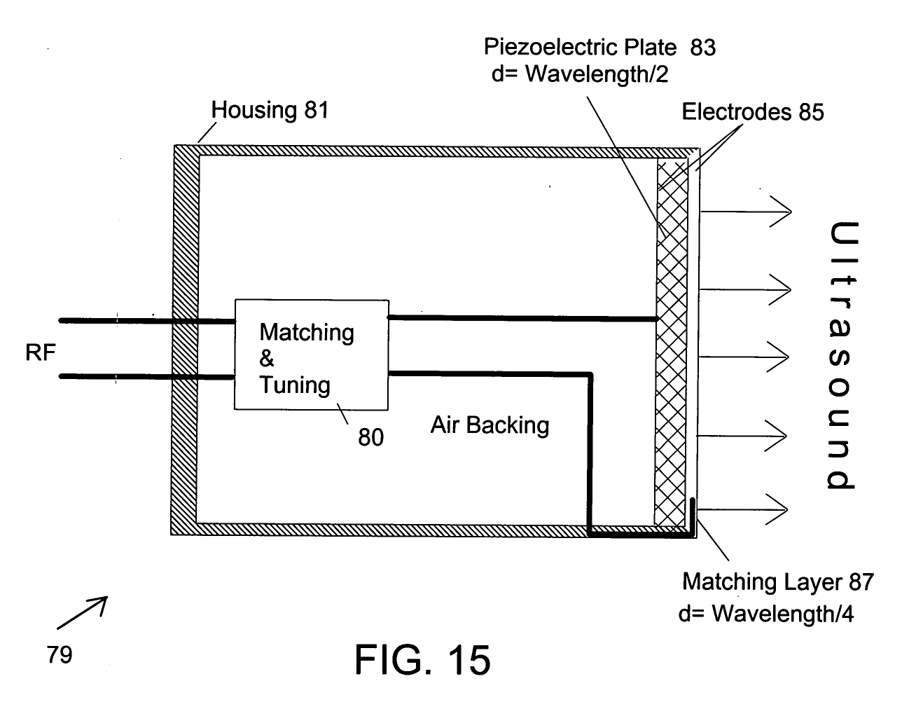

Shown in conjunction with FIG. 15 is a general structure of a high-power ultrasound transducer. The thickness of the plate of piezoelectric material 83 determines the operating frequency. Both surfaces of the transducer are covered by thin metal electrodes 85. The transducer plate is mounted on the holder in such a way that it has maximum freedom to move. On the front surface there can be a one-quarter wavelength matching layer 87 that reduces the acoustic mismatch between the transducer and the coupling media. However, it is optional and adequate power outputs can be obtained without it. An air space behind the plate provides a low impedance backing. This space can also house the electrical matching circuit 80. Maximum electrical efficiency of the transducer can be obtained when the transducer is matched to the electrical impedance of the driving amplifier and the electrical and mechanical resonances of the transducer are tuned together.

Piezoelectric materials lack a center of symmetry in their lattice structure, and have the property that the application of pressure causes an electrical voltage to appear across the crystal. The voltage is proportional to the applied pressure within the elastic limits of the material. By applying a changing voltage across a piezoelectric crystal, electrical energy can also be converted to mechanical thickness change of the crystal. As is known in the art, since hyperthermia transducers capable of producing high power, single-frequency continuous waves for extensive periods are needed, lead zirconate titante (PZT) is generally used. Also in reference to FIG. 15, the maximum stress wave is obtained when the thickness of the plate d=λ/2 or an odd multiple of λ/2. The frequency which corresponds to the half wavelength thickness is the fundamental resonant frequency of the transducer.

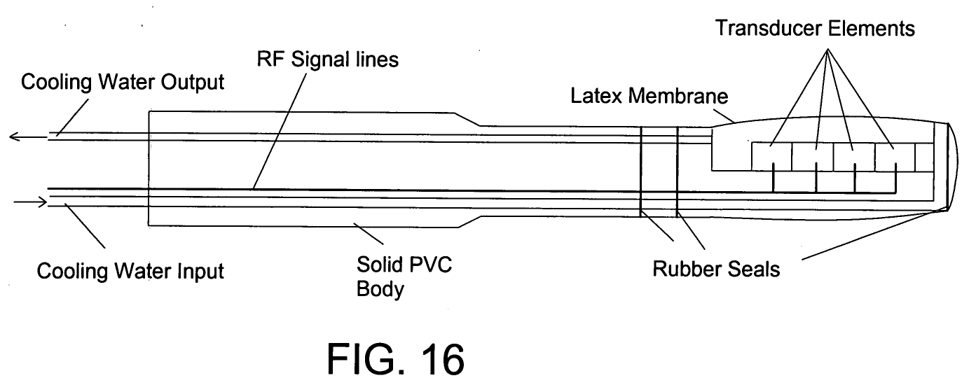

For the application of the current invention, the piezoelectric ceramic can be manufactured in the shape of a cylinder with electrodes on both inner and outer surfaces. When an RF voltage is applied on the electrodes, the cylinder wall thickness will expand and contract with the voltage. This generates a cylindrical ultrasound wave which propagates radially outward. Cylindrical applicators are known in the art for delivering for prostate applications, and can be similarly used for gastrointestinal (GI) applications of the current invention. One such four-element intracavitary applicator is shown in conjunction with FIG. 16. As will be clear to one of ordinary skill in the art, these can be adapted for the various gastric applications.

### Cryoablation

Cryoablation generally is a surgical technique that employs freezing to kill the target cells. The target tissue is frozen to a lethal temperature dependent on the tissue type to generate an ice ball. Accurate monitoring of the ice ball margin and temperature is achieved by employing intraoperative ultrasound and placing thermocouples inside of the cryoprobe.

The mechanism of tissue injury in cryoablation are not fully understood and there are some controversies about then. Generally, two mechanisms are considered as the main causes of direct cellular injury: (1) cell dehydration by osmosis when the ice ball is created in the extracellular space, and (2) intracellular ice formation at a high cooling rate.

At slow rates of cooling, tissues tend to freeze extracellularly. Slow cooling rates encourage the crystals to expand to a very large size. When these crystals develop in the extracellular space, migration of water out of the cells occurs because of the pressure gradients induced by the combined influence of concentration differences and capillarity. The ultimate end of such a process is dehydration of the cells and the development of external ice crystals which can be many times the size of individual cells.

At high cooling rate, the migration of water out of the cells may become inadequate to support the rapid growth of extracellular crystals. As a consequence, intracellular ice formation occurs, probably from growth of external ice through minute water-filled pores in the cell membrane. Intracellular ice crystals will tear down the membranes of cells and organelles inside the cell.

### Cryogen

Liquid nitrogen and argon are widely used as cryogens. The boiling temperatures of LN₂ and argon are −196° C. and −186° C., respectively. However, this low temperature is hard to attain in the probe design. One reason is back pressure, which limits the flow of cryogen into the cryoprobe, and the other reason is Liedenfrost boiling.

### Cryoprobe

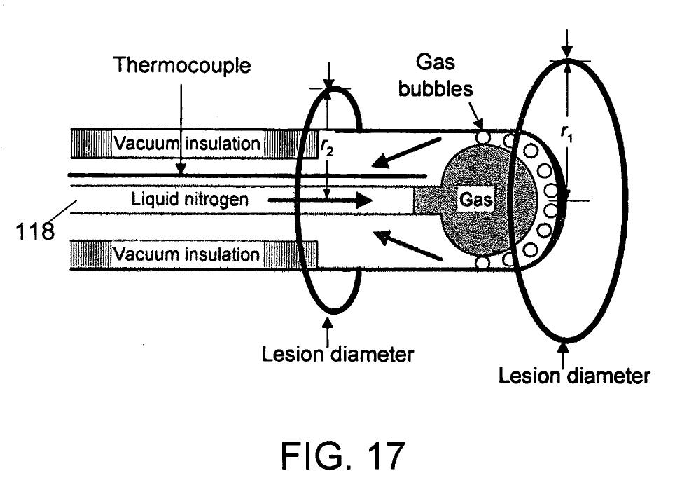

The LN₂ probe generally consists of a closed-end tube with two tubes concentrically arranged within it. Shown in conjunction with FIG. 17, is the basic design for a typical LN₂-based cryoprobe. Inside the probe, there is a funnel 118 for liquid nitrogen to go through. At the end of funnel, it hits the warm uninsulated tip of the cryoprobe where it changes phase, expanding 700 times in volume. The expanding gas exits the cryoprobe around the supply tube. This gas expansion is the constraint on the probe's functioning since it creates a back pressure that limits the flow of liquid nitrogen into the cryoprobe. Another phenomenon, caused by phase change, is the Liedenfrost boiling. When liquid nitrogen expands, gas bubbles form between the liquid and the metal, acting as an insulator. As a result, the temperature of the cryoprobe tip is about −160° C., not −196° C. The rate of complications and adverse effects are significantly higher with LN₂-based systems due to a slow response time to control adjustment.

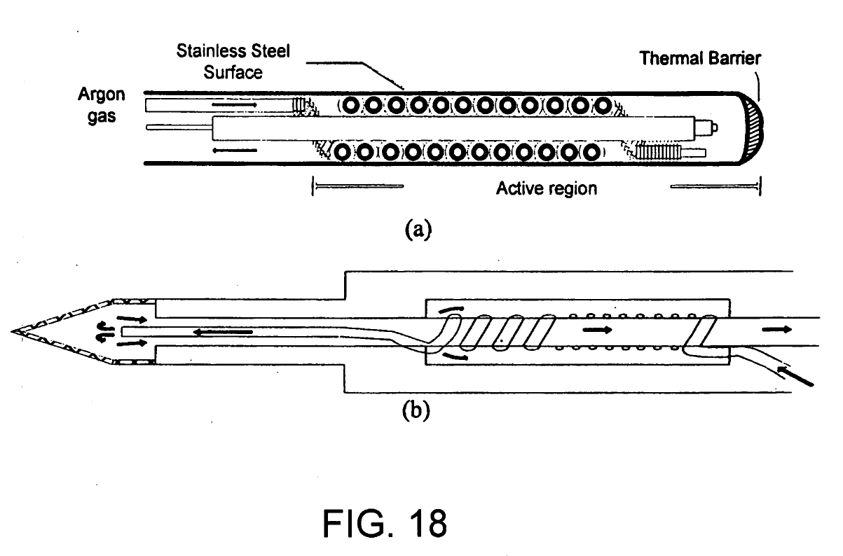

Shown in conjunction with FIG. 18 is an argon-based cryoprobe available from Endocare Inc., in which the system operation is based on the Joule-Thomson principle. Such a system can also be adapted for gastric ablation. In this system when a gas flows from a region of higher pressure to a region of lower pressure through a constricted passage (J-T port), it is said to be throttled. Based on Joule and Kelvin's principles, we know that most gases drop in temperature when throttled. For some gases, notably hydrogen and helium, the temperature rises. Whether there is a rise or fall in temperature depends on the particular range of pressures and temperatures over which the change occurs. For each gas, there are different values of pressure and temperature at which no temperature change occurs during a Joule-Thomson expansion. That temperature is the inversion temperature. The ratio of the observed drop in temperature to the drop in pressure-is-the-Joule-Thomson coefficient (dT/dP). The temperature of a particular gas increases or decreases after going through a J-T port depending on whether its original temperature is above or below its maximum inversion temperature. Generally, the temperature decreases as long as the maximum inversion temperature is above ambient temperature and vice versa.

FIG. 18(b) shows a different type of cryoprobe available from Galil Medical Ltd. The details of these systems are disclosed in U.S. Pat. No. 5,800,787 (a) and U.S. Pat. No. 6,142,991 (b), which are incorporated herein by reference.

### Laser Ablation

Lasers are widely used in ablations and many other medical applications, and can be adapted for use with gastric or other gastrointestinal (GI) ablations of the current invention. Lasers are coherent, and the energy of a laser beam is concentrated in a very narrow wavelength band. All photons in a laser beam are exactly in the same phase. Lasers are always directional. The direction of a laser beam is exactly parallel to the axis of the laser generator cavity. Lasers have these properties because of the way lasers are generated

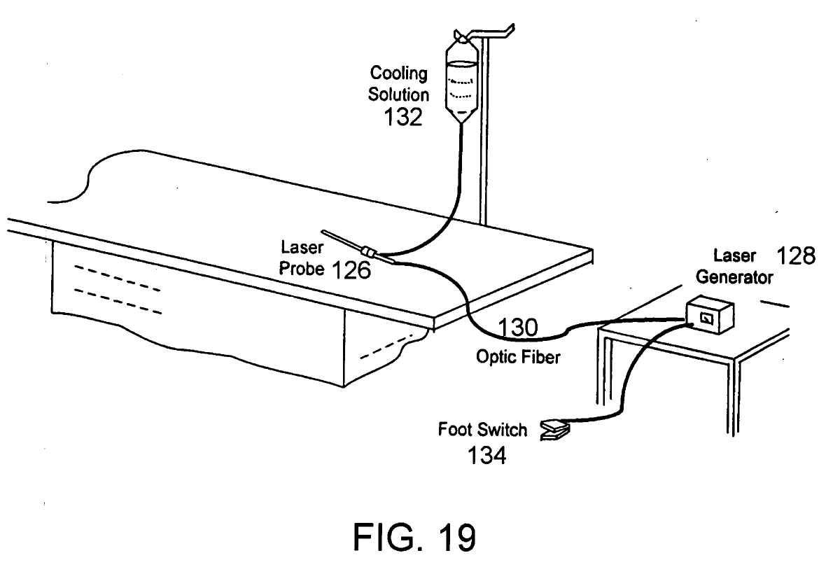

A typical laser ablation system is shown in conjunction with FIG. 19. The system consists of a solid state laser generator 128 with control system, an optical fiber cable 130, a laser probe 126, a water cooling system 132, and an external foot switch 134.

### Laser Tissue Ablation

With laser ablation, tissues are ablated through tissue coagulation, water vaporization, tissue dehydration, tissue cabonization and pyrolysis. Ablated tissue can be directly removed through vaporization and explosive mechanical ruptures.

### Laser System

Lasers are generated inside laser generator resonate cavities. The lasing medium could be a gas, dye, solid state crystal, or semiconductor. The excitation mechanism converts the electric power from the power supply unit to other types of energy to excite the lasing medium. After the lasing medium is excited, its molecules are energized from their low energy levels to their higher energy levels, which is the inverted population state. The lasing medium in its inverted population state emits free photons when its molecules transit from their higher energy levels back to their low energy levels. When free photons travel in the lasing medium and pass by other excited molecules, the excited molecules are stimulated to transit from their higher energy levels to their lower energy levels causing them to emit photons of the same frequency, phase, and direction as the free photons. This is the phenomenon of stimulated emission. Free photons are amplified by the stimulated emission effect in the laser generator cavity in all directions. Most of them will quickly exit from the cavity if they are not moving in a direction exactly parallel to the axis of the lasing cavity, and will be reflected back and forth between the high reflector and the output coupler, which is in fact, a partial reflector. Photons in the cavity-axis direction are reflected between the two reflectors. They are amplified by the stimulated emission effect of the excited lasing medium. Amplified photons form the unique phased and unidirectional output laser beam.

Compared to the complete reflector at one end of the cavity, the output coupler at the other end is actually a partial reflector. It lets the amplified laser photons partially exit from the laser cavity to become the output laser beam. The majority of the laser photons remain in the cavity to be further amplified by the excited lasing medium. The output coupler is usually connected with other optical delivery devices such as an optical fiber cable, which will conduct the output laser beam to the tissue where the laser beam will be applied.

The excitation mechanism excites the lasing medium and keeps it at its inverted population state. The excited lasing medium amplifies the laser beam in the cavity through the stimulation emission effect. The whole laser cavity is a balanced laser system as the electric power is taken from the power supply unit and converted to the output laser energy by the excitation mechanism and the lasing medium.

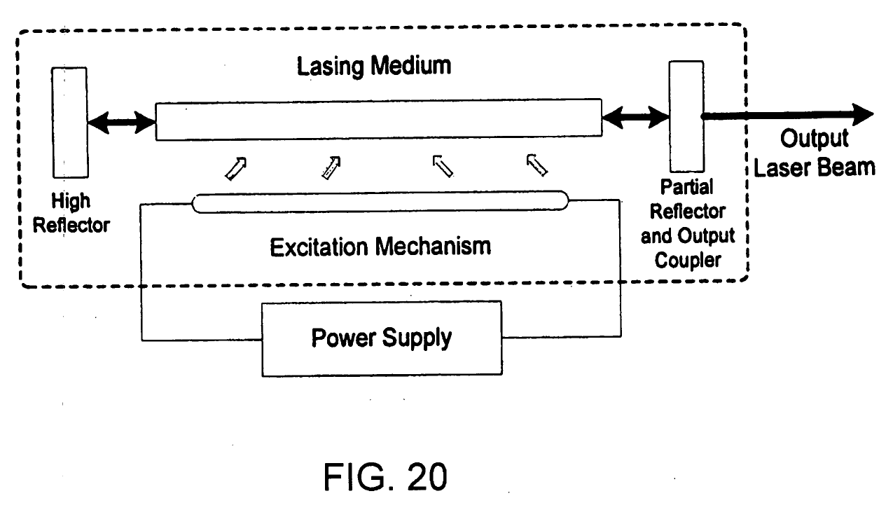

FIG. 20 shows a schematic diagram of a working laser generator, which can be adapted for gastric ablation application. The excitation mechanism is powered by the electric power supply unit and excites the lasing medium. Photons are amplified by the excited lasing medium and resonate between the high reflector and the output coupler. The output coupler releases a certain amount of laser photons to form the output laser beam.

### Laser Ablation Systems

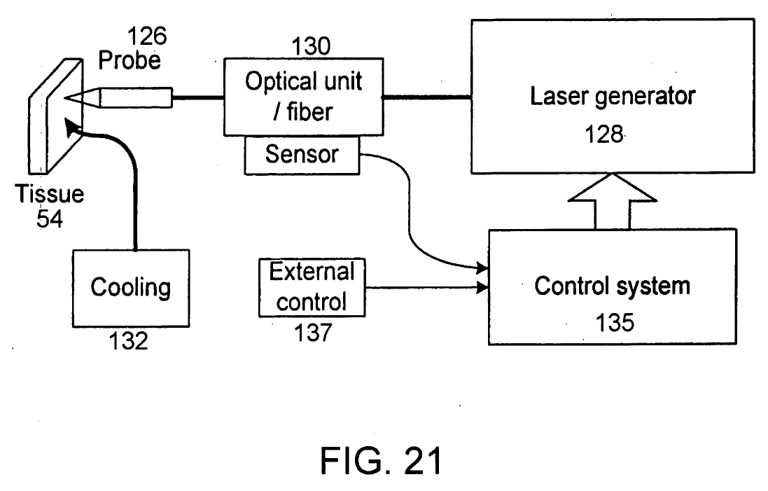

As shown in conjunction with FIG. 21, laser ablation systems usually consist of laser generator 128, computerized control systems 135, optical conductive units including optical fiber cables 130, laser probes 126. Lasers are generated by laser generators 128, which are controlled by their control systems 135. Output laser beams are coupled into optical delivery systems or optical fiber 130 cables and conducted to the laser probes 126. The laser probes then apply the laser beams to target tissues 54.

### Control Systems

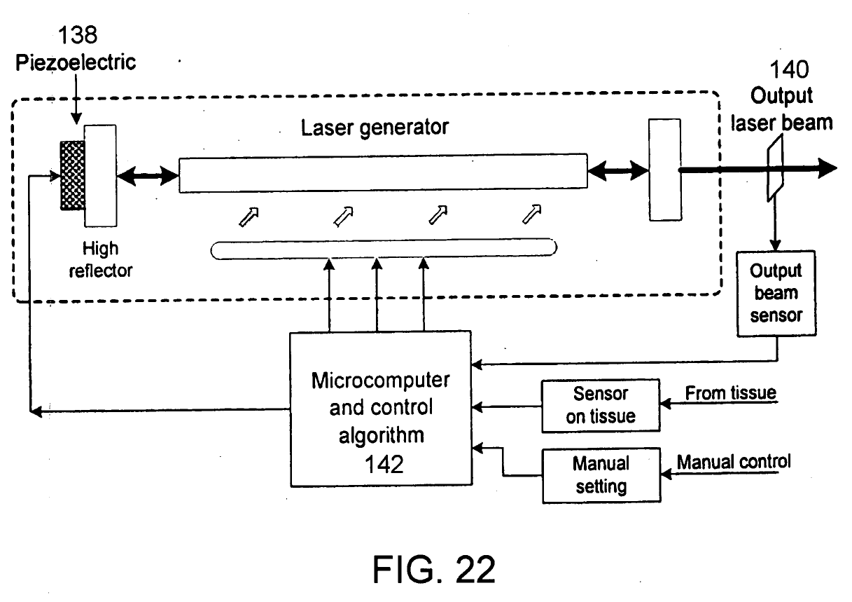

Control systems usually vary among different laser ablation systems. They are essential in controlling laser ablation procedures. Shown in conjunction with FIG. 22 is a generic control system. The microcomputer 142 with control algorithm is the center. The control center integrates inputs from different sensors and manual control settings, calculates optimized parameters by using these inputs according to preprogrammed control algorithms, and controls its effectors on the laser generator—the reflectors and the excitation mechanism. The generic control system shown in FIG. 22 can control laser output, pulse duration, and pulse frequencies by controlling the piezoelectic transducer 138, and it can control the laser output 140 pulse power densities by controlling the pumping mechanism.

The cooling systems effectively remove the heat which is generated when laser radiation energies are absorbed by target tissues. They reduce the amount of heat transferred to adjacent tissues, minimize the damage to the adjacent tissures, and improve the precision of ablation procedures. Both air spraying and water spraying are used as cooling mechanisms.

As is well known in the art, these systems can be adapted for gastric or other gastrointestinal (GI) tract ablations of the current invention.

### Gastrointestinal (GI) Ablations

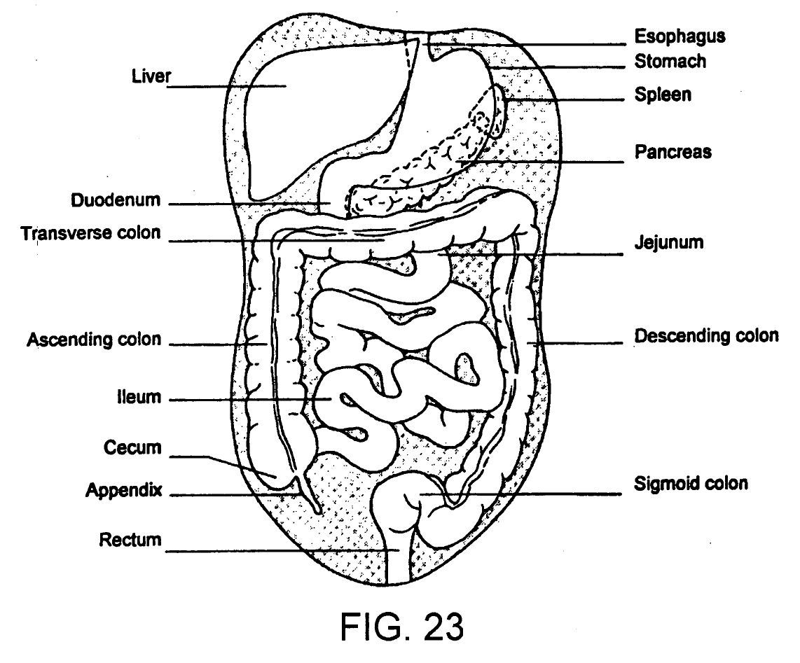

Shown in conjunction with FIG. 23 the gastrointestinal (GI) tract runs from the mouth to the anus and includes the esophagus, stomach, small bower or intestine, and the large bowel (colon) and rectum. The liver is also widely considered as a gastrointestinal organ.

In one object of the invention, the ablations may be performed as adjunct therapy for selected gastrointestinal abnormalities such as polyps, tumors, and cancers of the digestive system; specifically, the esophagus, stomach, liver, biliary tact, pancreas, colon, rectum, and anus.

### Liver Ablations in Stomach Cancer

Frequently in stomach cancer, the disease has already spread to the lymph system by the time of diagnosis. In such a case, the most common treatment is surgery by which cancer and surrounding stomach are removed. Cryoablation, and radiofrequency ablation, and/or standard liver resection may be performed at the time of stomach resection, depending on physician judgement.

### Ablation Therapies for Hepatic Colorectal Carcinoma Metastases

Although hepatic resection remains the gold standard for the treatment of live tumors, a large number of patients are not ammenable to surgical therapy. This may be due to unfavorable anatomy, the presence of multiple tumors, or poor hepatic reserve. Cryoablation, RF ablation, and laser ablation may be applied for the ablation of liver tumors.

### Hepatic Tumor Ablations

Hepatic cancer is one of the most common malignancies. It has two primary types: hepatocellular carcinoma (tumor starts in the liver) and metastatic (tumor originates elsewhere and spreads to the liver). A majority of patients are not amenable to hepatic resection surgery due to the size, location, and number of tumors. Several ablative techniques are used instead.

For patients with one to three small tumors located near the surface of the liver, laparoscopic or percutaneous RF treatments yield good results. Using ultrasonic guidance, a needle probe is inserted through the skin and into the tumor. The tip of the probe opens to expose an umbrella shaped array of hook electrodes once on site. RF power is applied for 5 to 15 min, and the current design is capable of producing lesions approximately 3 cm in diameter. The major factor limiting the lesion size comes from hepatic perfusion. By performing this minimally invasive surgery, fewer patients suffer from side effects, such as infection, bile leakage, or breathing difficulties, and most reoccurrences of cancer are along the outer edges of tumors that are too large to be completely destroyed.

RF ablation during an open operation is more suitable for patients with numerous, larger tumors, or tumors located near large blood vessels. More accurate placement of the probe can be achieved in open surgery, therefore increasing the attainable lesion size. Some side effects, such as bleeding of the parenchymal cells, can be avoided as well. Compared with other ablative technologies, particularly cryablation, RF ablation yields a relatively low complication rate and a lower overall mortality. It has the worst performance when applied to tumors located near blood vessels since the nearby blood flow significantly convects away the heat. Other disadvantages include long procedure time and difficulty in ultrasonic imaging of the lesion.

### Localized Gastrointestinal Carcinoid Tumor

In one aspect, if the regional disease is found to be unresectable, palliative surgery such as cryoablation or RF ablation may be performed. Treatment is customized for each patient depending on the growth of the tumor and/or symptoms.

### Tissue Ablation—Obesity

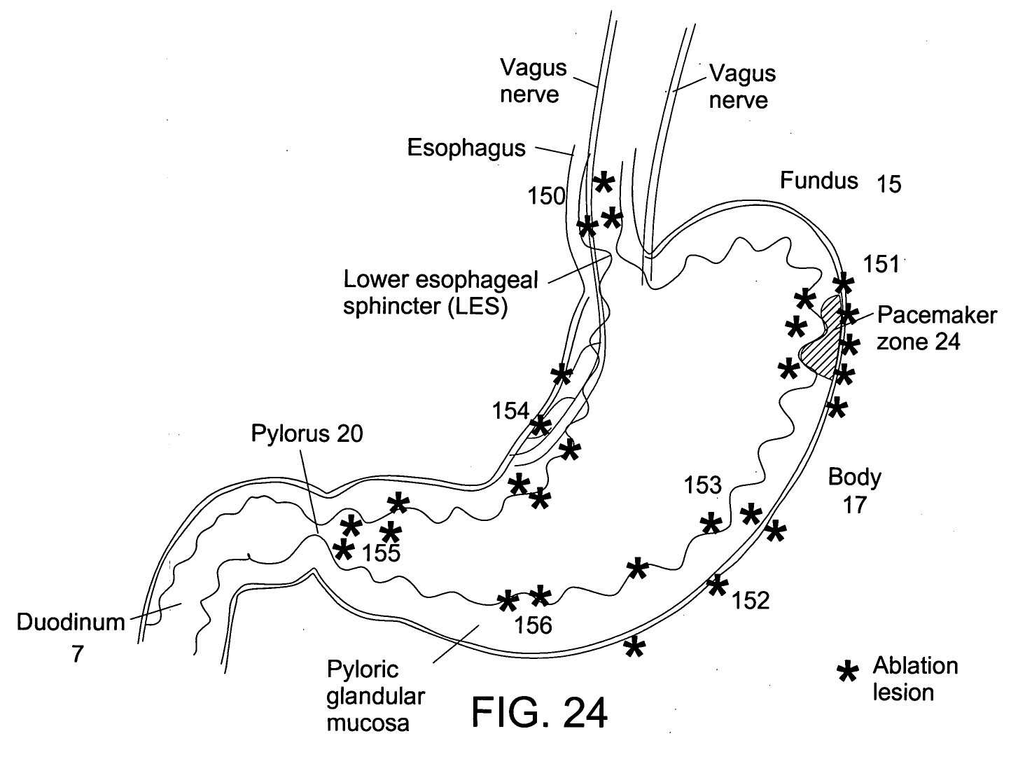

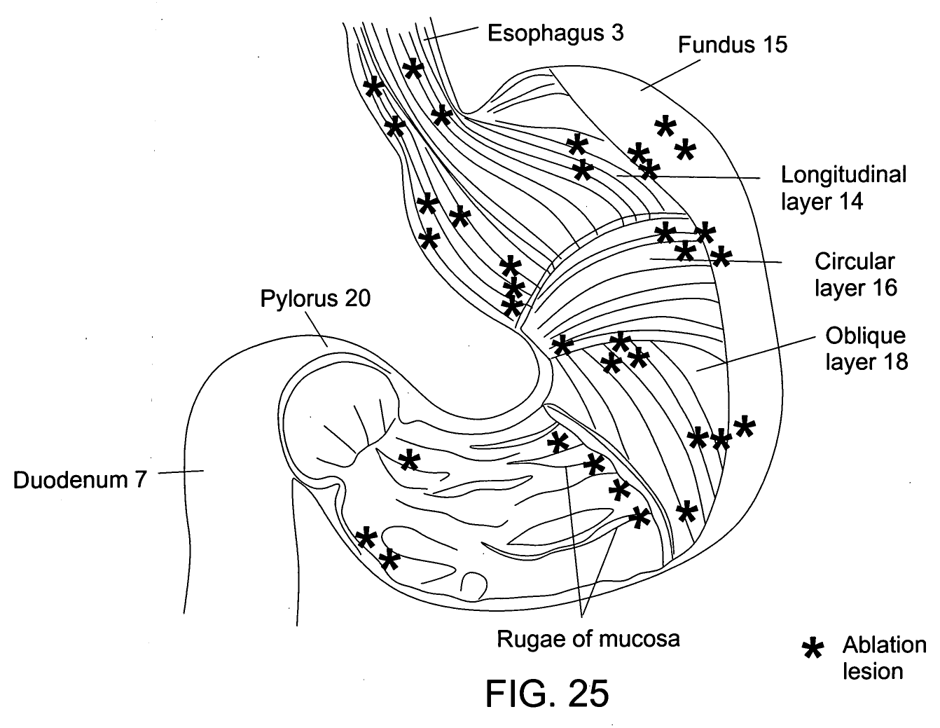

Shown in conjunction with FIGS. 24 and 25 are sites marked where ablations may be performed as a treatment for obesity or to induce weight loss. As was previously mentioned, the ablations may be approached from the epigastric side via laproscopic surgery, or may be approached via the mouth and esophagus in an anesthetized patient, and be performed endogastrically. Also, as previously mentioned the ablations may be performed using radiofrequency catheter ablation, radiofrequency catheter ablation using irrigated tip catheter, microwave ablation, high intensity focused ultrasound (HIFU) ablation, cryoablation, or laser ablation.

### Disruption of Pacemaker Region of the Stomach

Various mechanisms which disrupt the normal physiology of stomach and the associated nervous system may be targeted for ablation, whereby disrupting the normal function which leads to weight loss. For example, ablating the pacemaker region of the stomach can slow normal electrical activity of stomach.

Under normal circumstances, the pacesetter cells, which are smooth muscle cells that are capable of rhythmic, autonomous, partial depolariztion, are located in the upper fundus 15 region of the stomach. These cells generate slow-wave potentials that sweep down the length of the stomach toward the pyloric sphincter at a rate of approximately three per minute. Depending on the level of excitability in the smooth muscle, they may initiate contractions recognized as peristaltic waves that sweep over the stomach in pace with the basic electrical rhythm (BER) at a rate of 3/minute. By ablating at and around the pacemaker region, the intent is to decrease basic electrical rhythm (BER), whereby the stomach empties less efficiently, which leads to a feeling of “fullness”, and the patient's do not feel hungry. As shown with reference to FIG. 24, the ablation around the pacemaker zone 25 may be from the inside, or outside of the stomach, or both.

Applicant's patent application Ser. No. 11/047,233 and Ser. No. 11/032,298 disclose accomplishing a similar function by stimulating the gastric muscle. The advantage of ablation is that no hardware component needs to be left in the body. Another advantage of ablation is that it may offer a more permanent solution, without concern for battery status of an implantable device.

### Ablation of Gastric Muscle

In another object of the invention, the ablation lesions may be performed with the intent of disrupting the muscle layers of gastric wall. In this disclosure, the terms stomach, stomach muscle, gastric wall, and gastric wall muscle are used interchangeably. Shown in conjunction with FIG. 24, are 3 layers of stomach, which are the longitudinal layer 14, the circular layer 16, and the oblique layer 18. As shown in conjunction with FIGS. 24 and 25 ablation may be performed at various regions of the stomach. In FIG. 24, sites 152, 153, 154, and 155 are just some examples. The ablation lesion may be performed in clusters or individual lesions may be connected to form lines, or a combination of lines and clusters. In this aspect, even though the basic electrical rhythm (number of contractions) does not change, the contractility of the gastric muscle does become less efficient, which leads to longer times for the stomach to empty, thereby generally providing a feeling of fullness, and the patient feeling less hungry or not hungry.

### Ablation of Endogastric Lining—for Cells that Produce the Hunger Hormone Ghrelin

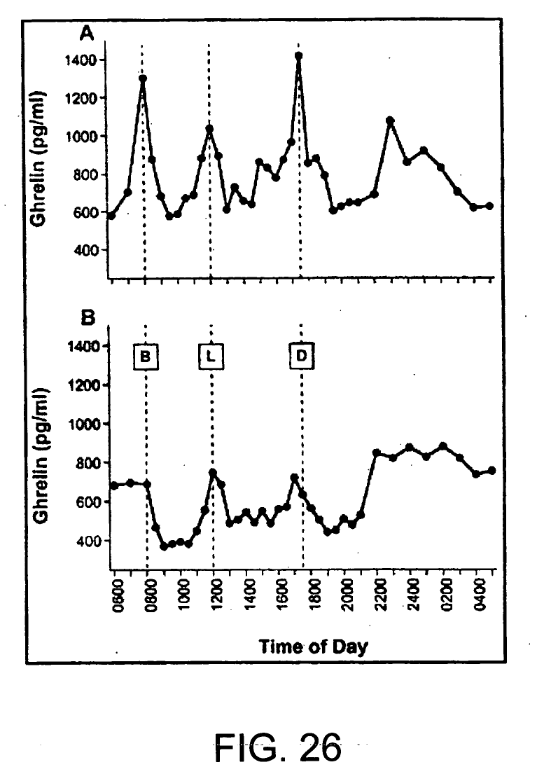

In another object of the invention, ablation may be performed to damage the mucosal cells lining the stomach that secrete the hormone ghrelin. The hormone ghrelin has been termed the appetite hormone. As shown in conjunction with FIG. 26, the levels of ghrelin typically have peaks which correspond to breakfast, lunch, and dinner, labeled B, L, and D respectively. The rationale for the ablation is that by ablating the mucosal cells, the plasma levels of ghrelin will decrease, leading to appetite suppression and weight loss.

### Disruption of Enteric Nervous System

In another object of the invention, ablation is performed for disruption of parts of the enteric nervous system. This is also shown in conjunction with FIGS. 24 and 25. In FIG. 24, ablation sites marked as 152, 153, 154 and 156 will also disrupt the functioning of the enteric nervous system, when the lesions are deep enough in the muscle. This can be approached from the endogastric side or epigastric side.

The stomach is richly innervated by extrinsic nerves and by the neurons of the enteric nervous system. Axons from the cells of the intramural plexus innervate smooth muscle and secretory cells. Parasympathetic innervation to the stomach is also supplied by the vagus nerves, while sympathetic innervation to the stomach is provided by the celiac plexus. In general, parasympathetic nerves stimulate gastric smooth muscle motility and gastric secretions, whereas sympathetic activity inhibits these function. Numerous sensory afferent fibers leave the stomach in the vagus nerves; some of these fibers travel with sympathetic nerves. Other sensory neurons are the afferent links between sensory receptors and the intramural plexuses of the stomach. Some of these afferent fibers relay information intragastric pressure, gastric distention, intragastric pH, or pain.

By disrupting the enteric nervous system, the functioning of stomach is made less efficient. This will also lead to inefficient emptying of the stomach, and a general feeling of fullness. In other words, the patient will not feel as hungry

### Vagal Nerve(s) Disruption

In one object of the invention, the ablation may be targeted to vagal nerve(s) disruption. Again, this may be done from the endogastric side (via mouth and esophagus) or from the epigastric side. Shown in conjunction with FIG. 24, these area's are labeled 150 around the esophagus, and 154 on the stomach diagram. As one example, without limitation, a probe connected to high intensity focused ultrasound (HIFU) source may be placed in the distal end of the esophagus, close to lower esophageal sphincter (LES), and ablation of vagal nerve(s) may be performed through the esophagus. Alternatively, microwave energy source may be used, and the site marked 154 which is around the lesser curvature of the stomach may be targeted. Clinical studies have confirmed that surgical vagotomy promotes weight loss.

### Ablation for Gastroesophageal Reflex Disease (GERD)

The methods and system disclosed, may also find use in gastroesophageal reflux disease (GERD). GERD results from the chronic backward flow of stomach contents into the esophagus. The acid, bile, and digestive enzymes cause irritation of the esophagus and symptoms of heartburn, regurgitation, chest pain, voice disorders, and swallowing problems.

Normally, the muscular valve (lower esophageal sphincter or LES) at the junction of the esophagus and stomach prevents reflux from occurring. Reflux of stomach contents occurs when the LES and diaphragm are unable to provide enough tone or force to squeeze adequately on the esophagus. This may happen in some patients in whom the muscles have weakened over time or in those patients with hiatal hernias. The barrier function in these patients is completely lost, and reflux is present throughout the day.

The majority of patients with GERD, however, have normal LES and diaphragm pressures, yet the sphincter muscles relax frequently throughout the daytime to cause reflux. The relaxation events permit excessive reflux of stomach contents and the patient develops significant symptoms of GERD.

This abnormal event is a neurological reflex, which is the transient lower esophageal relaxation (tLESR) and is the cause of GERD in over 80% of patients. A tLESR is prompted when there is stretching of the stomach wall, as after a meal. The stretch receptors generate a nerve impulse, which travels upward within the myenteric plexus of the gastroesophageal junction. The myenteric plexus is a network of very small nerves lying between the layers of the stomach and esophagus musculature. The impulses travel through the LES, into the esophagus, and then join the vagus nerve on their way to the brain. When the brain receives these signals, a motor signal is sent to the LES causing prolonged relaxation.

The importance of tLESR in the development of GERD has been extensively investigated. Clinical investigations have also focused on the delivery of radiofrequency energy for the treatment of GERD.

Investigators at Stanford University have recently performed radiofrequency ablation of the stomach cardia in Yucatan mini-pigs to establish the effect on these nerve pathways. These nerve fibers course between the muscle layers of the LES and cardia. The effect of delivering radiofrequency energy to the cardia on the parameter of gastric yield pressure has been shown. This test is directly related to tLESRs. The stomach is stretched with carbon dioxide gas until the LES yields or relaxes in response to pressure. Yield pressures were higher in all animals after treatment, indicating that the nerve reflex arc was modulated to have a higher threshold for stimulation, or a lower frequency of transmission to the brain.

In one preferred embodiment, ablation therapy is performed for treating reflux disease, as is shown in conjunction with FIGS. 27A and 27B. As depicted in FIG. 27A, a catheter utilizing a balloon, may be used. Shown in conjunction with FIGS. 27A and 27B, the balloon may be deflated or inflated.

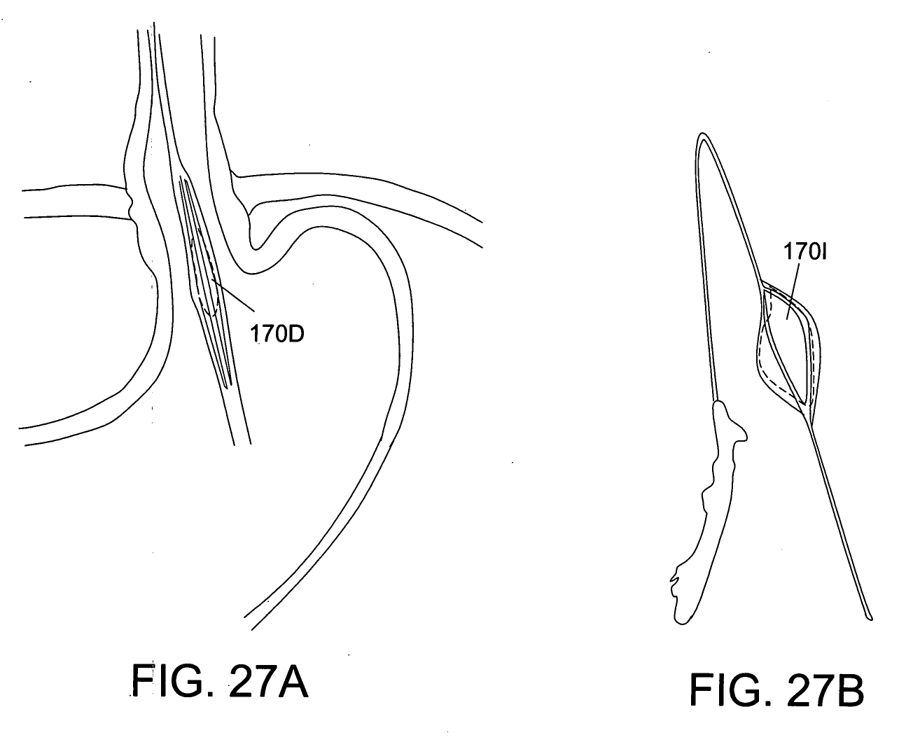

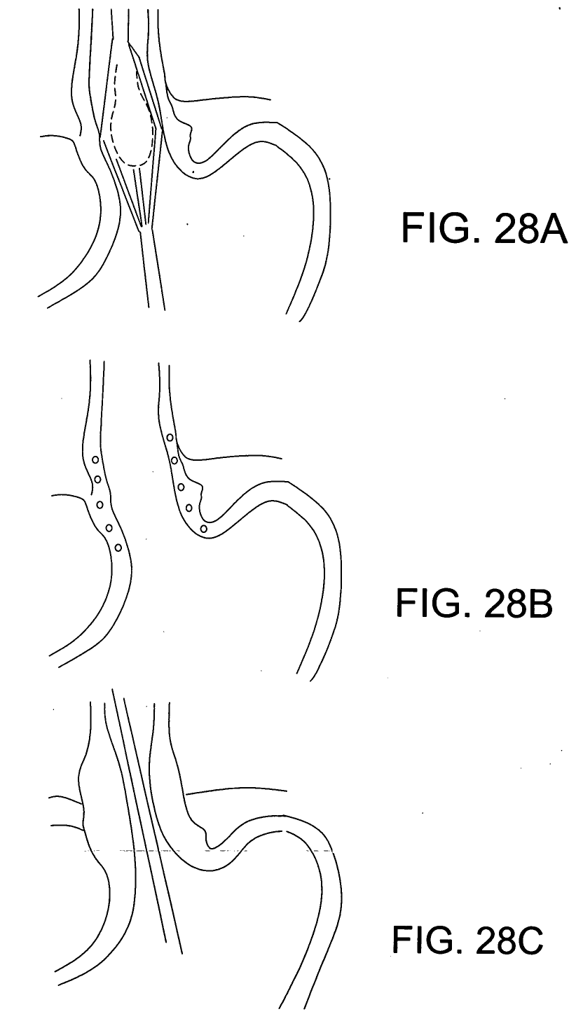

Shown in conjunction with FIGS. 28A, 28B, and 28C, the physician positions the catheter, inflates the balloon, deploys the needles and begins irrigation, During treatment, radiofrequency energy is delivered in a controlled manner to the tissue surrounding the needle electrodes (FIG. 28A). The treatment sequence is repeated to create well-defined coagulative lesions along the length of the lower esophageal sphincter and cardia (FIG. 28B). Over the next few weeks, the coagulated tissue resorbs and shrinks, increasing resistance to reflux (FIG. 28C).

Many embodiments of the invention have been described. Various modifications may be made without departing from the scope of the claims. It is therefore desired that the present embodiment be considered in all aspects as illustrative and not restrictive, reference being made to the appended claims rather than to the foregoing description to indicate the scope of the invention.

What is claimed is:

1. A method of providing selective ablations to part(s) of gastrointestinal (GI) tract for treating or alleviating the symptoms of at least one of gastrointestinal tumors, obesity, motility disorders, GERD, or to induce weight loss, comprising the steps of:
a) selecting a patient for said selective ablation;
b) selecting one or more sites for said selective ablation;
c) selecting one or more kind of ablation means to provide said selective ablation;
d) selecting the intensity of ablations for said one or more sites; and
e) providing said ablation by at least one of radiofrequency (RF) catheter ablation means, radiofrequency ablation with irrigated catheter means, microwave ablation means, high intensity focused ultrasound ablation means, cryoablation means, and laser ablation means.

2. The method of claim 1, wherein said radiofrequency energy is delivered between approximately 300 KHz and 1,000 K Hz.

3. The method of claim 1, wherein said microwave is provided at approximately 915 MHz or approximately 2,450 MHz.

4. The method of claim 1, wherein said ablations are directed to gastric muscle.

5. The method of claim 1, wherein said ablations are directed to gastric muscle and nerve tissue.

6. A method of selectively ablating portions of gastrointestinal (GI) tract or liver to provide adjunct (add-on) therapy or alleviate symptoms for at least one of gastrointestinal tumors, obesity, motility disorders, GERD, or to induce weight loss, with radiofrequency catheter ablation or microwave ablation, comprising the steps of:
a) selecting a patient to be ablated;
b) selecting portions of gastrointestinal (GI) tissue to be ablated in said patient; and
c) providing said ablation by one of radiofrequency catheter ablation means comprising, a radiofrequency ablation generator, a ground patch connected to patient and said radiofrequency ablation generator, and a catheter adapted for reaching said portions of gastrointestinal (GI) tract to be ablated; or microwave ablation means, comprising a catheter antenna adapted for reaching portions of gastrointestinal (GI) tract being ablated, and a microwave ablation generator, wherein said microwave ablation generator further comprises a microwave source, coupling network, and a power supply.

7. The method of claim 6, wherein said radiofrequency energy is delivered between approximately 300 KHz and 1,000 K Hz.

8. The method of claim 6, wherein said microwave energy is provided at approximately 915 MHz or approximately at 2,450 MHz.

9. The method of claim 6, wherein said ablation is applied to a gastric wall.

10. The method of claim 6, wherein said ablation is directed to gastric mucosa.

11. The method of claim 6, wherein said ablation of said gastric wall may use epigastric approach or endogastric approach.

12. The method of claim 6, wherein said ablation may involve ablation to nerve tissue around the stomach.

13. The method of claim 12, wherein said nerve tissue may be part of enteric nervous system.

14. The method of claim 12, wherein said nerve tissue may be part of vagus nerve tissue, or branches, or parts thereof.

15. The method of claim 6, wherein said ablation is applied to a endogastric lining of the stomach wall.

16. A method of selectively ablating portions of gastrointestinal (GI) tract or liver to provide adjunct (add-on) therapy or alleviate symptoms for at least one of gastrointestinal tumors, obesity, motility disorders, GERD, or to induce weight loss, with high intensity focused ultrasound (HIFU) ablation, comprising the steps of:
a) selecting a patient to be ablated;
b) selecting portions of gastrointestinal (GI) tissue to be ablated in said patient; and
c) providing an ultrasound applicator adapted for applying ultrasound energy to said gastrointestinal (GI) tissue to be ablated; and
d) providing an electromechanical component means for steering and positioning acoustic beam, a display therapy planning and imaging, and a computer for HIFU dosage calculation, control and for monitoring feedback during ablation.

17. The method of claim 16, wherein said ablation is for ablating vagus nerve(s), or branches or parts thereof.

18. The method of claim 16, wherein said ablation is applied in the esophagus, close to a lower esophageal sphincter (LES).

19. The method of claim 16, wherein said ablations are applied close to lesser curvature of the stomach.

20. The method of claim 16, wherein said ablation are applied with a frequency of around 5 MHz.
